(19) United States
(12) Patent Application Publication
(10) Pub. No.: US 2026/0347479 A1
(43) Pub. Date: Nov. 5, 2026

(54) LENGTH-ADAPTED CHIMERIC ANTIGEN RECEPTORS AND LENGTH-ADAPTED ACCESSORY RECEPTORS FOR IMPROVED CAR T CELL ACTIVATION

(71) Applicant: Oxford University Innovation Limited, Oxford (GB)

(72) Inventor: Omer Dushek, Oxford (GB)

(73) Assignee: Oxford University Innovation Limited, Oxford (GB)

(21) Appl. No.: 19/101,998

(22) PCT Filed: Aug. 9, 2023
(86) PCT No.: PCT/GB2023/052105

§ 371 Date: Feb. 7, 2025

(30) Foreign Application Priority Data
Aug. 9, 2022 (GB) 2211637.0

(51) Int. Cl. C07K 16/28; A61K 40/11; A61K 40/31; A61K 40/42; C07K 14/705; C12N 5/0783

(52) U.S. Cl. CPC C07K 16/2833; A61K 40/11; A61K 40/31; A61K 40/4211; A61K 40/4213; C07K 14/70507; C07K 14/70517; C07K 14/70521; C07K 16/2803; C12N 5/0636; C07K 2317/53; C07K 2317/55; C07K 2317/622; C07K 2319/02; C07K 2319/03; C12N 2510/00

(57) ABSTRACT

The present invention relates to optimising the effector function of an immune effector cell that expresses a chimeric antigen receptor (CAR) the co-localises with an accessory receptor, by ensuring that the intermembrane distance spanned by the CAR-antigen complex is comparable with the intermembrane distance spanned by the accessory receptor-ligand complex.

### FIELD OF INVENTION

The invention relates to chimeric antigen receptors (CARs) and accessory receptors and methods for optimising effector functions when said CARs and/or accessory receptors are expressed in immune effector cells.

### BACKGROUND OF THE INVENTION

T cells patrol the body in search of antigens derived from infectious organisms or cancer cells. They use their T cell antigen receptors (TCRs) to recognise peptide antigens on major-histocompatibility-complexes (pMHC). T cells have remarkable antigen sensitivity; they can become activated when recognising only a single pMHC. This high sensitivity is important because infectious organisms and cancers deploy evasion mechanisms to reduce the amount of antigen presented to T cells.

An exciting new treatment for cancer is to re-program a patient's T cells to target their cancer. This is done by using genetic engineering to express chimeric antigen receptors (CARs) on T cells. They allow a patient's T cells to recognise and kill their cancer cells. This therapy is approved to treat B cell cancers. However, many patients relapse with B cells that express low levels of the target antigen. It is now clear that CARs have a profound defect in antigen sensitivity; CARs require 100-1000-fold more antigen than the TCR to activate T cells. The mechanisms underlying the defect in CAR antigen sensitivity had not been elucidated.

There have been many attempts to improve CARs with a focus on its intracellular signalling domain and/or targeting multiple different antigens. The inventors previously found that the defect in CAR sensitivity can be primarily attributed to the failure of CARS to exploit accessory receptors (1).

There is an urgent need to increase the sensitivity of CARs to prevent cancer relapses. More sensitive CARs would also allow CAR T cells to be used in treating a wider variety of cancers.

It is therefore an object of the invention to provide further and improved CARs and/or accessory receptors, thereby improving CAR T cell therapy.

### SUMMARY OF THE INVENTION

The inventors found that optimal antigen recognition by CARs requires optimal alignment between the membrane of the immune effector cell and the membrane of the antigen presenting cell (APC). Membrane alignment is influenced by the dimensions of receptor-ligand complexes, such as CARs and accessory receptors that co-localise on the immune effector cell and their respective antigens and ligands on the APC. Interestingly, the inventors found that antigen recognition by a CAR is optimised when the intermembrane distance spanned by the complex between the CAR and the target antigen (CAR-antigen complex) is comparable with the intermembrane distance spanned by the complex between an accessory receptor that co-localises with the CAR and its ligand (accessory-ligand complex). Hence, the invention provides CARs and/or accessory receptors of appropriate sizes for optimisation of membrane alignment, and these CARs and accessory receptors of the invention are also referred to herein as variable size CARs (vsCARs) and variable size accessory receptors, respectively.

In particular, the inventors showed that modulating the size of the extracellular portion of the CAR, and hence the height of the extracellular antigen binding domain of the CAR, resulted in varying antigen sensitivity, as demonstrated by the modulation of T cell effector function. Example 1 shows that a CAR prepared according to conventional designs in the art, which uses a CD8a or CD28 hinge between the extracellular antigen binding domain and the transmembrane domain, exhibited poor antigen sensitivity. On the other hand, a CAR that has a smaller hinge, and hence having a shorter extracellular antigen binding domain from the membrane of the immune effector cell, compared to the CAR prepared according to conventional designs, exhibited good antigen sensitivity, comparable to that of a T cell receptor (TCR) and a synthetic T cell receptor and antigen receptor (STAR) but higher than the conventional CAR and an epsilon-T cell receptor fusion construct (εTRuC). This shows that extracellular dimensions, rather than intracellular signalling, optimises antigen recognition. Furthermore, the smaller CAR does not associate with the TCR-CD3 complex, which is an advantageous feature for the purpose of clinical use.

The inventors also showed that modulating the size of the extracellular portion of the accessory receptor, and hence the height of the extracellular ligand binding domain of an accessory receptor, e.g. CD2, from the membrane of the immune effector cell resulted in varying antigen sensitivity of the CAR, as demonstrated by modulation of T cell effector function. Example 2 shows that the antigen sensitivity of a CAR prepared according to conventional designs in the art was improved by increasing the height of the extracellular ligand binding domain of an accessory receptor (e.g. CD2). Hence, engineering accessory receptor-ligand complexes by varying the dimensions of their extracellular portion maximises their ability to enhance CAR-antigen complexes.

As shown in FIG. 1A, the accessory receptor-ligand complex spans an intermembrane distance (e.g. 14 nm for the CD2-CD58 complex) compatible with TCR-pMHC complex. However, this intermembrane distance is not optimised for a CAR prepared according to conventional designs in the art (CAR_{CON}, i.e. a CAR comprising a CD28 hinge or a CD8a hinge) and the target antigen. The usefulness of the vsCARs and variable size accessory receptors of the invention are explained below.

For example, as shown in FIG. 1B, where the intermembrane distance spanned by a complex of a CAR_{CON} and the target antigen is larger than the intermembrane distance spanned by the accessory receptor-ligand complex, CAR signalling would be weak (FIG. 1B, left). To improve CAR signalling, a vsCAR which is smaller than the CAR_{CON} can be used, such that the vsCAR-antigen complex spans an intermembrane distance that is compatible with the accessory receptor-ligand complex (e.g. 14 nm for the CD2-CD58 complex (2,3)). Hence optimal membrane alignment is achieved (FIG. 1B, right). Alternatively, as shown in FIG. 1C, optimal membrane alignment and hence improvement in antigen recognition, can also be achieved by using a variable size accessory receptor that is larger than the corresponding endogenous accessory receptor, e.g. elongated CD2. Hence, the variable size accessory receptor-ligand complex spans an intermembrane distance that is compatible with the CAR_{CON}-antigen complex.

As a further example, as shown in FIG. 1D, in a situation where the target antigen is large making it difficult to optimise membrane alignment using the endogenous accessory receptor-ligand (e.g. CD2-CD58) complex because even a vsCAR of a small size may span an intermembrane distance too long for the formation of a complex with the target antigen, a vsCAR can be paired with a variable size accessory receptor to optimise membrane alignment, and hence improve antigen recognition and CAR signalling.

The variable size accessory receptors can be prepared by modifying the size of the stalk between the extracellular ligand binding domain and the transmembrane domain, which consequently determines the height of the extracellular ligand binding domain of the accessory receptor from the membrane of the immune effector cell. For example, as shown in FIG. 1E, various sequences that physically increase the height of the ligand binding domain from the membrane of the immune effector cell (also referred to herein as an inflexible spacer) can be used. Thus, the invention provides an accessory receptor comprising a variable size stalk to control the height of the extracellular ligand binding domain of the accessory receptor for optimising effector function of an immune effector cell elicited by a CAR, wherein the accessory receptor and the CAR are expressed on said immune effector cell.

The vsCARs can be prepared by modifying the size of the hinge between the antigen binding domain and the transmembrane domain, which consequently determines the height of the extracellular antigen binding domain of the CAR from the membrane of the immune effector cell. For example, as shown in FIG. 1F, various sequences that physically increase the height of the ligand binding domain from the membrane of the immune effector cell (also referred to herein as an inflexible spacer) can be used. Thus, the invention provides a CAR comprising a variable size hinge to control the height of the extracellular antigen binding domain of the CAR for optimising effector function of the immune effector cell in which the CAR is expressed.

Furthermore, the inventors have overcome a significant problem in the field of CAR technology. In particular, CARs that have entered the clinic and/or proved successful in clinical trials are typically directed to membrane-proximal epitopes expressed on an APC when compared to the antigenic site of peptide-MHC complexes. This has allowed such CARs to exploit endogenous accessory receptors, such as the CD2-CD58 complex, to enhance T cell activation. The inventors have now provided a mechanism by which membrane-distal antigens expressed on an APC may also be successfully targeted by CARs, such as peptide-MHC complexes themselves or larger antigens, whilst retaining the effect achieved by accessory receptors to enhance T cell activation.

Accordingly, the invention provides an immune effector cell comprising:
a chimeric antigen receptor (CAR) capable of binding to an antigen on an antigen presenting cell (APC), wherein the CAR comprises a fusion protein comprising an extracellular antigen binding domain, a transmembrane domain, and an intracellular signalling domain, wherein the extracellular antigen binding domain and the transmembrane domain are linked by a hinge; and
an accessory receptor capable of binding to a ligand on the APC, wherein the accessory receptor comprises an extracellular ligand binding domain and a transmembrane domain, wherein the extracellular ligand binding domain and the transmembrane domain are linked by a stalk;
wherein:
(a) the accessory receptor is an exogenous accessory receptor and the stalk comprises a sequence that physically increases the height of the extracellular ligand binding domain from the membrane of the immune effector cell;
(b) the hinge of the CAR comprises or consists of a sequence that physically increases the height of the antigen binding domain from the membrane of the immune effector cell, wherein the sequence comprises a mucin-like sequence, one or more folded polypeptide domains or a fragment of the CD28 as set out in SEQ ID NO: 10 or the CD8a hinge as set out in SEQ ID NO: 43; and/or
(c) the CAR and the accessory receptor are each of a size such that the intermembrane distance spanned by the CAR-antigen complex is comparable with the intermembrane distance spanned by the accessory receptor-ligand complex.

The invention also provides a chimeric antigen receptor (CAR) capable of binding to an antigen on an antigen presenting cell (APC), comprising a fusion protein comprising an extracellular antigen binding domain, a transmembrane domain, and an intracellular signalling domain, wherein the extracellular antigen binding domain and the transmembrane domain are linked by a hinge, wherein the hinge comprises or consists a sequence that physically increases the height of the extracellular antigen binding domain from the membrane of the immune effector cell, wherein the sequence comprises a mucin-like sequence, one or more folded polypeptide domains or a fragment of the CD28 as set out in SEQ ID NO: 10 or the CD8a hinge as set out in SEQ ID NO: 43.

The invention also provides an accessory receptor capable of binding to a ligand on the APC, comprising an extracellular ligand binding domain and a transmembrane domain, wherein the extracellular ligand binding domain and the transmembrane domain are linked by a stalk, wherein the stalk comprises or consists of a sequence that physically increases in the height of the ligand binding domain from the membrane of the immune effector cell, optionally wherein the stalk comprises a mucin-like sequence.

The invention also provides an immune effector cell comprising or encoding the CAR and/or accessory receptor of the invention.

The invention also provides a method of preparing the immune effector cell, comprising introducing a nucleic acid encoding a CAR and/or an accessory receptor of the invention into an immune effector cell.

The invention also provides a method of optimising the effector function of an immune effector cell, wherein the immune effector cell comprises:
a chimeric antigen receptor (CAR) capable of binding to an antigen on an antigen presenting cell (APC), wherein the CAR comprises a fusion protein comprising an extracellular antigen binding domain, a transmembrane domain, and an intracellular signalling domain, wherein the extracellular antigen binding domain and the transmembrane domain are linked by a hinge; and
an accessory receptor capable of binding to a ligand on the APC, wherein the accessory receptor comprises an extracellular ligand binding domain and a transmembrane domain, wherein the extracellular ligand binding domain and the transmembrane domain are linked by a stalk;
wherein the method comprises modifying the height of the extracellular antigen binding domain of the CAR and/or the height of the extracellular ligand binding domain of the accessory receptor from the membrane of the immune effector cell to optimise the effector function of the immune effector cell upon contact with the APC.

The invention also provides a method for identifying an improved immune effector cell, wherein the immune effector cell comprises:
a chimeric antigen receptor (CAR) capable of binding to an antigen on an antigen presenting cell (APC), wherein the CAR comprises a fusion protein comprising an extracellular antigen binding domain, a transmembrane domain, and an intracellular signalling domain, wherein the extracellular antigen binding domain and the transmembrane domain are linked by a hinge; and
an accessory receptor capable of binding to a ligand on the APC, wherein the accessory receptor comprises an extracellular ligand binding domain and a transmembrane domain, wherein the extracellular ligand binding domain and the transmembrane domain are linked by a stalk;
wherein the method comprises:
(a) modifying the height of the extracellular antigen binding domain of the CAR and/or the height of the extracellular ligand binding domain of the accessory receptor from the membrane of the immune effector cell; and
(b) determining whether an immune effector cell expressing a modified CAR and/or a modified accessory receptor has improved effector function when compared to an immune effector cell expressing an unmodified CAR and/or unmodified accessory receptor.

The invention also provides a method for identifying an improved immune effector cell, comprising determining whether the immune effector cell of the invention has improved effector function when compared to an immune effector cell expressing an unmodified CAR and/or unmodified accessory receptor.

The invention also provides an immune effector cell obtained or obtainable by a method of the invention.

The invention also provides a method, such as an ex vivo method, of preparing a population of immune effector cells for adoptive cell therapy, the method comprising culturing the immune effector cell of the invention to produce a population of immune effector cells.

The invention also provides a population of immune effector cells produced by a method of the invention.

The invention also provides a method for treating cancer in a subject, the method comprising administering an effective amount of the immune effector cell or the population of immune effector cells of the invention to the subject.

The invention also provides the immune effector cell or the population of immune effector cells of the invention for use in a method of treating cancer in a subject.

The invention also provides the use of the immune effector cell or the population of immune effector cells of the invention in the manufacture of a medicament for the treatment of cancer.

The invention also provides the use of the immune effector cell or the population of immune effector cells of the invention to treat cancer.

The invention also provides the use of the immune effector cell or the population of immune effector cells of the invention for adoptive cell therapy.

### BRIEF DESCRIPTION OF THE FIGURES

FIG. 1. Schematic of the mechanism for optimising antigen sensitivity by membrane alignment. (A) The accessory receptor-ligand complex (e.g. CD2-CD58) spans an intermembrane distance (e.g. about 14 nm) compatible with TCR/pMHC binding. (B) In a situation where the size of a standard CAR is not compatible with the size of the accessory receptor-ligand complex (e.g. CD2-CD58) (left), a hinge of appropriate size can be selected to be inserted in the CAR to match this distance (right). For example, a variable size CAR (vsCAR) can be used such that the vsCAR-antigen complex spans an intermembrane distance compatible with the accessory receptor-ligand complex, and hence optimal membrane alignment is achieved thereby improving antigen recognition. (C) Alternatively, a variable size accessory receptor, e.g. elongated CD2, can be used such that the variable size accessory receptor-ligand complex spans an intermembrane distance compatible with the conventional CAR-antigen complex, and hence the conventional CAR achieves an optimal alignment with target antigen thereby improving antigen recognition. (D) In a situation where the target antigen is large making it difficult to optimise membrane alignment using the naturally occurring accessory receptor-ligand (e.g. CD2-CD58) complex because even a vsCAR may span an intermembrane distance too large for the wild-type complex (left), a vsCAR can be paired with a variable size accessory receptor to optimise membrane alignment (right). (E) Design of variable size accessory receptor. The accessory receptor may be varied in size using an inflexible spacer consisting of various numbers of amino acid residues (e.g. 4, 8, 20, 40 or 234 amino acid residues) taken from CD43 (e.g. any of SEQ ID NOs: 50 to 62). See for example elongated CD2 variants as set out in SEQ ID NOs: 27 to 36. (F) Design of vsCAR. The shortest CAR contains a ‘mini’, ‘micro’, ‘nano’ or truncated hinge from the therapeutic CD28 CAR (see e.g. SEQ ID NOs: 37, 41 and 42). This CAR can be varied in size using an inflexible spacer consisting of various numbers of amino acid residues (e.g. 4, 8, 20, 40 or 234 amino acid residues) taken from CD43 (e.g. any of SEQ ID NOs: 50 to 62). For example, see SEQ ID NOs: 38 to 40, which may be applied to hinges other than the mini-CD28 hinge.

FIG. 2. Schematic of 1G4 T cell receptor (“TCR”), D52N CAR (“CAR”), D52N Fab CAR (“Fab CAR”) and an IgG1 antibody. 1G4 TCR comprises a variable alpha domain and constant alpha domain, a P2A self-cleaving peptide, followed by a variable beta domain and constant beta domain. The 1G4 TCR uses the endogenous CD3 components. D52N CAR is a CAR prepared according to conventional designs with the D52N single-chain variable fragment (scFv). D52N is an antigen binding domain that recognises the same peptide antigen that is recognised by the 1G4 TCR. The conventional design includes, for example, a D52N scFv followed by a hinge and transmembrane domain (e.g. CD28 or CD8a), and an intracellular signalling domain (e.g. CD28 cytoplasmic domain and a zeta chain (CD247) cytoplasmic domain). The D52N Fab CAR is a CAR of the invention. It comprises the variable heavy domain of the D52N scFv, coupled to IgG1-CH1 and the variable light domain of the D52N scFv coupled to IgG1-CL domain, and each of the light and heavy domains is coupled to a CD28 transmembrane domain, a CD28 cytoplasmic domain, and a zeta chain (CD247) signalling tail.

FIG. 3. Schematics of antigen receptors: TCR (1G4), STAR (D52N), ε-TRUC (D52N), Fab CAR (D52N Fab-28z) and CAR (D52N-CD28-28z). The TCR (1G4), Fab CAR (D52N Fab-28z) and CAR (D52N-CD28-28z) are as described in FIG. 2. The antigen receptors all recognise the same peptide antigen (SLLMWITQV; SEQ ID NO: 66) presented on HLA-A*02:01. In the case of the TCR, the variable domains of the 1G4 TCR are used whereas for the other receptors tested, the D52N variable domains are used which are derived from the 3M4E5 antibody (4,5).

FIG. 4. Surface CD3E and surface antigen receptor expression profiles in TCRα⁻ β⁻ Jurkat cells transduced with the 1G4 TCR, a D52N-CD28-28z CAR and a D52N-Fab CAR-28z receptor. Staining was conducted on E6.1 TCRα⁻β⁻ cells Jurkat cells lentivirally transduced with the constructs indicated. Tetramers of the pMHC antigen detects surface levels of each antigen receptor (y-axis) and UCHT antibody detects surface CD3ε levels. The CAR and Fab CAR constructs are detected at the cell surface without CD3ε upregulation indicating these receptors are CD3-independent, which is not the case for the 1G4 TCR.

FIG. 5. Expression profiles of the TCR (1G4), STAR (D52N), ε-TRUC (D52N), the D52N-Fab CAR and a D52N-CD28-28z CAR. Staining was conducted on primary human CD8+ T cells that, after lentiviral transduction with the corresponding receptor, were subject to antibiotic selection.

FIG. 6. Primary CD8+ T cell activation expressing different antigen receptors assessed by flow cytometry. T cells were incubated with the T2 target cell line loaded with different concentrations of the NY-ESO-1 9V peptide antigen for 20 hours before assessing the surface activation markers (A) 4-1BB and (B) CD25. (C) The EC50 from three independent donors. The EC50 is the concentration of antigen required to elicit 50% of the maximum response. Statistical analysis was conducted on the log-transformed value of the EC50 for the 4-1BB and CD25 readouts, using Dunnett's multiple comparisons test (ns=not significant, *=p<0.05, **=p<0.01, ***=p<0.001).

FIG. 7. Primary CD8+ T cell activation expressing different antigen receptors assessed by cytokine production. T cells were incubated with the T2 target cell line loaded with different concentrations of the NY-ESO-1 9V peptide antigen for 20 hours before assessing the cytokine IFN-g (top row) and IL-2 (bottom row) by ELISA. Data is shown from three independent donors.

FIG. 8. The antigen sensitivity of CARs can be improved by varying the size of the hinge. (A) Schematics of a conventional CAR with the CD28 hinge (SEQ ID NO: 10) and a panel of variable size CARs with the indicated hinges. The CD28 mini hinge contains a smaller hinge compared to the conventional CAR (SEQ ID NO: 37). Larger hinges are produced by including the indicated size spacers from CD43 (see SEQ ID NOs: 38 to 40). (B) Primary human CD8+ T cells expressing the indicated antigen receptors are co-cultured with the Nalm6 target cell pulsed with the indicated concentration of peptide antigen. The surface expression of the 4-1BB activation marker is measured after 5 hours. (C) The fitted EC50 for the indicated antigen receptor.

FIG. 9. Schematic of the mechanism for optimising antigen sensitivity by membrane alignment using variable size accessory receptors. (A) Design of a variable size accessory receptor. In the present work, the ligand binding domain of the accessory receptor is the ectodomain of CD2 and is elongated with fragments of the mucin-like sequence of the extracellular portion of CD43. The transmembrane and intracellular domain is taken from wild-type CD2. (B) Schematic of experimental system. Jurkat TCRα⁻β⁻ T cells, which do not express CD8, were subjected to CRISPR to remove endogenous CD2. Jurkat TCRα⁻β⁻ CD2⁻ were transduced with an antigen receptor before being transduced with CD2 WT or different variable size accessory receptors using the design in (A).

FIG. 10. Wild-type CD2 optimises the antigen sensitivity of the T cell receptor. (A) Schematic of antigen receptor. (B, C) Surface CD69 on T cells co-cultured with U87 target cell lines pulsed with the indicated concentration of peptide antigen. Representative dose-response (B) and EC50 from multiple experiments (C). The arrow indicates the CD2 that optimises antigen sensitivity.

FIG. 11. Wild-type CD2 optimises the antigen sensitivity of the STAR. (A) Schematic of antigen receptor. (B, C) Surface CD69 on T cells co-cultured with U87 target cell lines pulsed with the indicated concentration of peptide antigen. Representative dose-response (B) and EC50 from multiple experiments (C). The arrow indicates the CD2 that optimises antigen sensitivity.

FIG. 12. CD2-CD43 (40) optimises the antigen sensitivity of the D52N-CD8a-z CAR. (A) Schematic of antigen receptor. (B, C) Surface CD69 on T cells co-cultured with U87 target cell lines pulsed with the indicated concentration of peptide antigen. Representative dose-response (B) and EC50 from multiple experiments (C). The arrow indicates the CD2 that optimises antigen sensitivity.

FIG. 13. CD2-CD43 (20) optimises the antigen sensitivity of the D52N-CD28-28z CAR. (A) Schematic of antigen receptor. (B, C) Surface CD69 on T cells co-cultured with U87 target cell lines pulsed with the indicated concentration of peptide antigen. Representative dose-response (B) and EC50 from multiple experiments (C). The arrow indicates the CD2 that optimises antigen sensitivity.

FIG. 14. CD2-CD43 (20) optimises the antigen sensitivity of the ε-TRUC (D52N). (A) Schematic of antigen receptor. (B, C) Surface CD69 on T cells co-cultured with U87 target cell lines pulsed with the indicated concentration of peptide antigen. Representative dose-response (B) and EC50 from multiple experiments (C). The arrow indicates the CD2 that optimises antigen sensitivity.

FIG. 15. Elongating the CD2-CD58 complex impairs antigen recognition for TCR (top) but it improves antigen recognition for CARs (bottom) based on sub-optimal and optimal membrane alignment, respectively.

FIG. 16. A. Jurkat T cells were transduced with the FM63-CD8a-41BBz (Kymriah) chimeric antigen receptor. Surface levels were detected by GFP-Spycatcher fused to Spytag-CD19. B. Kymriah-expressing Jurkat T cells were transduced with variable size CD2 molecules and sorted for matched expression using anti-CD2 PE. C. Nalm6 CombiCells expressing Spycatcher were coupled with purified Spytag-CD19 at different concentrations and detected by flow cytometry. D. Representative dose-response for Kymriah CAR Jurkat T cells with the indicated variable size CD2 molecule. E. Summary measure of antigen sensitivity (EC50) across N=3 independent experiments. A t-test is used to determine p-values for the null hypothesis that the EC50 is the same between the CD2 WT and the other CD2 condition with a Sidak-Holm correction for multiple comparisons. Abbreviations: *=p-value≤0.05, **=p-value≤0.01, ***=p-value≤0.01.

### DETAILED DESCRIPTION OF THE INVENTION

### Chimeric Antigen Receptors (CARs)

An immune effector cell of the invention comprises a chimeric antigen receptor (CAR). A CAR is a non-naturally occurring protein that comprises an extracellular portion comprising an antigen binding domain specific for an antigen, and the antigen binding domain is linked, via hinge and transmembrane domains, to an intracellular portion comprising one or more signalling moieties.

An immune effector cell of the invention may comprise a CAR of the invention. In this embodiment, the immune effector cell may also comprise an accessory receptor of the invention.

An immune effector cell of the invention may comprise a CAR prepared according to conventional designs, also referred to herein as conventional CAR or CAR_{CON}, e.g. as described in Reference 6, which comprises a CD28 hinge as set out in SEQ ID NO: 10 or a CD8a hinge as set out in SEQ ID NO: 43. In this embodiment, the immune effector cell also comprises an accessory receptor of the invention.

A CAR of the invention has improved antigen sensitivity when compared to a conventional CAR. A CAR of the invention is of a size such that the intermembrane distance spanned by the CAR-antigen complex is comparable with the intermembrane distance spanned by accessory receptor-ligand complex. The size (e.g. height) of the extracellular portion of a CAR, in particular, the height of the extracellular antigen binding domain of the CAR from the membrane of the immune effector cell, is important in determining antigen sensitivity. The optimal size (e.g. height) of the extracellular portion of a CAR, in particular, the optimal height of the extracellular antigen binding domain of the CAR from the membrane of the immune effector cell, is dependent on the sizes (e.g. heights) of the target antigen, the accessory receptor and its ligand, as explained further below.

A CAR of the invention may comprise an extracellular antigen binding domain having a height that is of comparable size (e.g. within ≤5% or ≤10% of, or is the same as) with the height of the extracellular antigen binding domain of a T cell receptor from the membrane of an immune effector cell.

The extracellular antigen binding domain of a CAR of the invention may have a height of about 7 nm from the membrane of an immune effector cell.

A CAR of the invention may comprise an extracellular antigen binding domain having a height that is taller than the extracellular antigen binding domain of a T cell receptor from the membrane of an immune effector cell, e.g. by ≤15%, ≤20%, ≤25%, or ≤30%.

The extracellular antigen binding domain of a CAR of the invention may have a height taller than about 7 nm but shorter than about 47 nm from the membrane of an immune effector cell. The extracellular antigen binding domain of a CAR of the invention may have a height of >7 nm, ≥10 nm, ≥15 nm, ≥20 nm, ≥25 nm, ≥30 nm, ≥35 nm, ≥40 nm, ≥45 nm from the membrane of an immune effector cell. The extracellular antigen binding domain of a CAR of the invention may have a height of ≤45 nm, ≤40 nm, ≤35 nm, ≤30 nm, ≤25 nm, ≤20 nm, ≤15 nm, or ≤10 nm from the membrane of an immune effector cell.

A CAR of the invention may comprise an extracellular antigen binding domain having a height that is shorter than the extracellular antigen binding domain of a T cell receptor from the membrane of an immune effector cell, e.g. by ≤15%, ≤20%, ≤25%, or ≤30%.

The extracellular antigen binding domain of a CAR of the invention may have a height taller than about 3 nm but shorter than about 7 nm from the membrane of an immune effector cell. The extracellular antigen binding domain of a CAR of the invention may have a height of ≥3 nm, ≥4 nm, ≥5 nm or ≥6 nm from the membrane of an immune effector cell. The extracellular antigen binding domain of a CAR of the invention may have a height of <7 nm, ≤6 nm, ≤5 nm, or ≤4 nm from the membrane of an immune effector cell.

The height of the extracellular antigen binding domain of a CAR of the invention from the membrane of an immune effector cell may be determined according to routine methods in the art, as described herein.

The hinge between the antigen binding domain and the transmembrane domain may determine the size (e.g. height) of the extracellular portion, in particular, the height of the extracellular antigen binding domain, of a CAR of the invention. The inventors found that conventional CARs have poor antigen sensitivity. This was improved by modulating (e.g. increasing or decreasing) the size of the hinge of the CAR, allowing optimal membrane alignment.

Hence, a CAR of the invention comprises a hinge of a different size compared to a conventional CAR, such that the CAR of the invention may have a smaller or larger extracellular portion, and as a consequence a shorter or taller extracellular antigen binding domain than a conventional CAR.

A CAR of the invention does not comprise a hinge consisting of: (a) the CD8a hinge as set out in SEQ ID NO: 43 or (b) the CD28 hinge as set out in SEQ ID NO: 10.

The hinge of a CAR of the invention comprises or consists of a sequence that physically increases the height of the antigen binding domain from the membrane of the immune effector cell.

The sequence may be an inflexible sequence, e.g, wherein each block of amino acid residues of the sequence physically increases the height of the antigen binding domain from the membrane of the immune effector cell because the sequence does not compress or fold back on itself. The block may comprise one, two, three, four, five, six, seven, eight, nine, ten or more amino acid residues.

The sequence may be a bulky sequence that physically increases the height of the antigen binding domain from the membrane of the immune effector cell, e.g. through steric hindrance, and thus prevents the antigen-binding domain from contacting the membrane.

The sequence may comprise or consist of a mucin-like sequence, one or more folded domains, or a fragment of the CD28 or CD8a hinge as set out in SEQ ID NOs: 10 and 43, respectively. The mucin-like peptide may be a fragment of the extracellular domain of CD43, as explained further below. The one or more folded domains may be one or more domains having an immunoglobulin fold, such as an immunoglobulin constant domain, or FNIII, as explained further below.

The sequence that physically increases the height of the antigen binding domain from the membrane of the immune effector cell may be of any suitable length. The sequence may be at least 1 amino acid in length, such as at least 5, at least 10 or at least 20 amino acids in length. The sequence may be 250 or fewer amino acids in length, 200 or fewer amino acids in length, 150 or fewer amino acids in length, or 100 or fewer amino acids in length, such as 80 or fewer, 60 or fewer, 40 or fewer, 30 or fewer, or 20 or fewer amino acids in length. The sequence may be 1 to 40 amino acids in length, such as 2 to 30, 3 to 25, 4 to 20, or 5 to 15 amino acids in length.

The sequence that physically increases the height of the antigen binding domain from the membrane of the immune effector cell may be an immunoglobulin domain, such as an immunoglobulin constant domain e.g. CH2 and CH3 region of IgG1, IgG2, IgG3, IgG4, CD8 (e.g. a CD8a hinge) or CD28. For example, when compared to a CAR_{CON}, a CAR of the invention may have the CD28 or CD8a hinge replaced with an immunoglobulin constant domain which is of a smaller size than the hinge.

In some cases, the sequence that physically increases the height of the antigen binding domain from the membrane of the immune effector cell is not an immunoglobulin domain.

The sequence that physically increases the height of the antigen binding domain from the membrane of the immune effector cell may be a fragment of the CD28 hinge as set out in SEQ ID NO: 10. The fragment preferably retains the cysteine residue at position 29 of SEQ ID NO: 10. Alternatively, the fragment may comprise a modification at position 29 of SEQ ID NO: 10 which removes the cysteine residue, e.g. by substitution. For example, the fragment may consist of ≤20 contiguous amino acids from the C-terminus end of SEQ ID NO: 10 or ≤30 contiguous amino acids from the N-terminus end of SEQ ID NO: 10. The fragment may consist of ≤5, ≤10, ≤15, ≤20, ≤25 or ≤30 contiguous amino acids from SEQ ID NO: 10 and containing the cysteine residue at position 29 of SEQ ID NO: 10. The fragment may consist of the sequence as set out in any one of SEQ ID NOs: 37, 41 or 42. For example, compared to a CAR_{CON} which contains the full length of SEQ ID NO: 10, a CAR of the invention may have a shorter CD28 hinge.

The sequence that physically increases the height of the antigen binding domain from the membrane of the immune effector cell may be a fragment of the CD8a hinge as set out in SEQ ID NO: 43. The fragment preferably retains the cysteine residue at position 27 of SEQ ID NO: 43. Alternatively, the fragment may comprise a modification at position 27 of SEQ ID NO: 43 which removes the cysteine residue, e.g. by substitution. For example, the fragment may consist of ≤30 or ≤25 contiguous amino acids from the C-terminus end of SEQ ID NO: 43 or ≤30 contiguous amino acids from the N-terminus end of SEQ ID NO: 43. The fragment may consist of ≤5, ≤10, ≤15, ≤20, ≤25 or ≤30 contiguous amino acids from SEQ ID NO: 43 and containing the cysteine residue at position 27 of SEQ ID NO: 43. The fragment may consist of the sequence as set out in any one of SEQ ID NOs: 46, 63 and 64. For example, compared to a conventional CAR which contains the full length of SEQ ID NO: 43, a CAR of the invention may have a shorter CD8a hinge.

A CAR of the invention may comprise one or more (e.g. ≥2, ≥3, ≥4, ≥5, ≥6, ≥7, ≥8, ≥9 or 10) immunoglobulin domains, such as immunoglobulin constant domains. A CAR of the invention may comprise no more than 10 immunoglobulin constant domains. The multiple immunoglobulin domains may be coupled in series or in parallel, for example where a CAR of the invention comprises more than one polypeptide, each polypeptide may comprise one or more immunoglobulin domains, such as ≥2, ≥3, ≥4 or ≥5 immunoglobulin domains.

The sequence that physically increases the height of the antigen binding domain from the membrane of the immune effector cell may be a mucin-like sequence, or fragments or derivatives thereof. Mucin like sequences are characterised by being rich in protein, serine and threonine residues, and the serine and threonine residues are heavily O-glycosylated. The mucin-like sequence may be a mucin-like sequence of the extracellular portion of CD43, i.e. as set out in SEQ ID NO: 62. Mucin-like sequences from other proteins may also be used, such as MUC1, MUC3A, MUC3B, MUC4, MUC12, MUC13, MUC15, MUC16, MUC17, MUC18, MUC20, MUC21, PSGL-1. Stalks of surface proteins that are mucin-like, such as CD8a and CD28, may also be used.

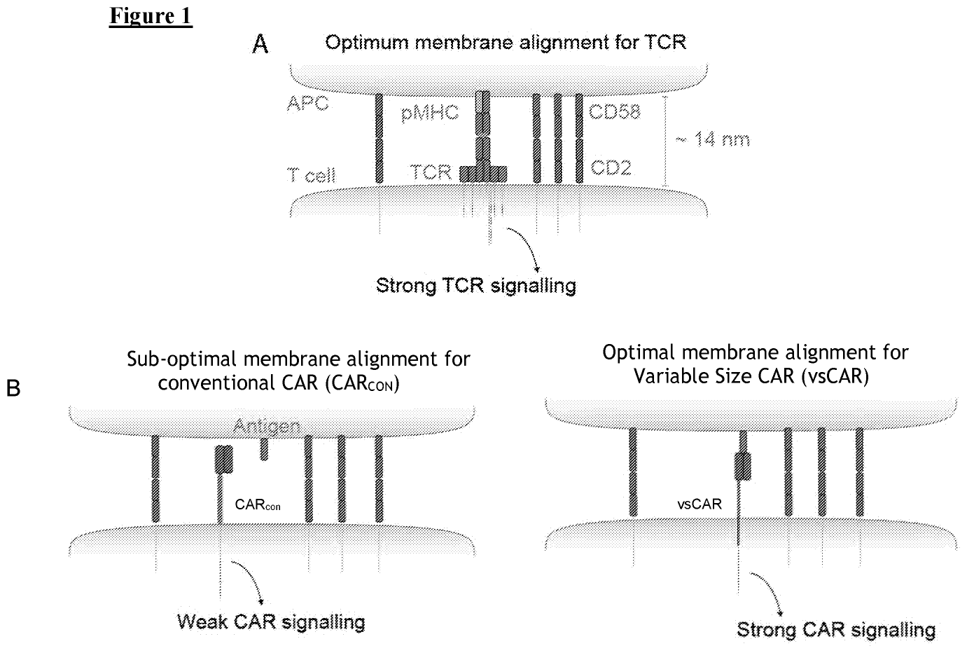

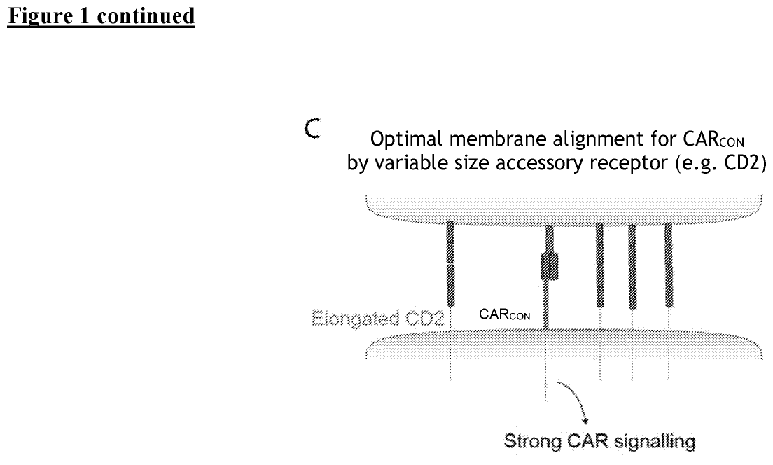

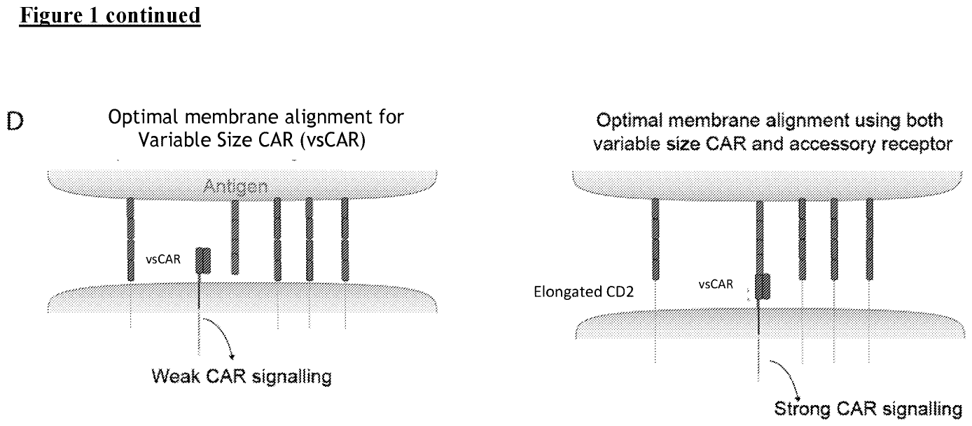

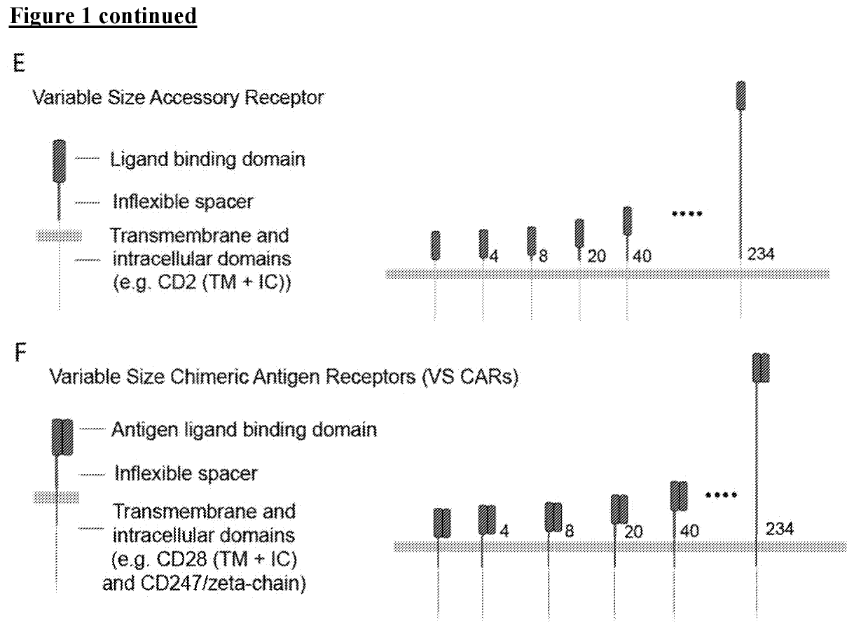

The mucin-like sequence may be a fragment of the extracellular domain of CD43, as set out in SEQ ID 62. For example, the fragment may be between 4 and 234 amino acids (e.g. as shown in FIG. 1F). The fragment may consist of ≥5, ≥10, ≥20, ≥30, ≥40, ≥50, ≥50, ≥70, ≥80, ≥90, ≥100, ≥110, ≥120, ≥130, ≥140, ≥150, ≥160, ≥170, ≥180, ≥190, ≥200, ≥210, ≥220, ≥230 contiguous amino acids from the N-terminus end or the C-terminus end of SEQ ID NO: 62. The fragment may consist of no more than 234 amino acids. The fragment may be between 4 and 120 amino acids, 8 and 80 amino acids, or 20 and 40 amino acids. The fragment may be any of SEQ ID NOs: 53 to 62.

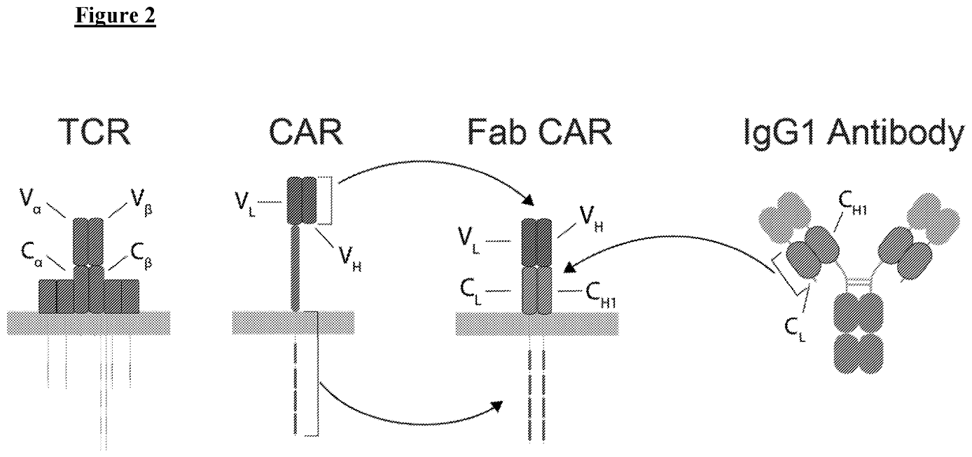

A CAR of the invention may be a dimer, such as a heterodimer. For example, the CAR may comprise two fusion proteins that form a dimer, and for each fusion protein, the antigen binding domain may be coupled to a folded polypeptide domain (e.g. an immunoglobulin constant domain), followed by a transmembrane domain (e.g. CD28 transmembrane domain) and an intracellular signalling domain (e.g. CD28 zeta-chain signalling domain). For example, the first fusion protein may comprise a light chain variable domain as the antigen binding domain and a light chain constant domain as the folded polypeptide domain, and the second fusion protein may comprise a heavy chain variable domain as the antigen binding domain and a heavy chain constant domain as the folded polypeptide domain. Such a CAR may effectively look like an antibody Fab coupled to a transmembrane domain (e.g. CD28 transmembrane domain) and an intracellular signalling domain (e.g. CD28 zeta-chain signalling domain). Hence, the extracellular portion of such CAR comprises two immunoglobulin constant domains (one from each chain) and two immunoglobulin light chains (one from each chain) and is comparable to the size (e.g. height) of a T cell receptor. For example, the CAR may be a Fab CAR as shown in FIG. 2. This allows an immune effector cell comprising such CAR to strongly interact with APCs displaying antigens that result in a CAR-antigen complex spanning an intermembrane distance of approximately 14 nm.

The antigen-binding domain of a CAR of the invention may be a scFv, a monoclonal antibody (comprising 2 heavy chains and 2 light chains), a polyclonal antibody, Fab, a Fab′, a F(ab′)2 fragment, a heavy chain variable domain (VH) or a nanobody (VHH). ScFv domains comprise a heavy chain variable domain (V-H) and a light chain variable domain (VL) of an immunoglobulin and connected by a short linker peptide.

A CAR of the invention may comprise more than one extracellular antigen binding domain, such as two extracellular antigen-binding domains or three extracellular antigen-binding domains. The two or more extracellular antigen binding domains may bind to different antigens, i.e. the CAR may be bispecific or multispecific.

The antigen binding domain of a CAR of the invention may be specific for any antigen, such as an antigen listed in Table 2. The antigen may be a peptide-MHC complex, CD19, mesothelin, BCMA, CD22, EGFR, or EGFRVIII. For example, the peptide of the peptide-MHC complex may be a NY-ESO-1 peptide, i.e. a peptide derived from proteolytic cleavage of NY-ESO-1.

The transmembrane domain of a CAR of the invention may be derived from a naturally occurring transmembrane protein, such as a type-I transmembrane protein. The transmembrane domain spans the cell membrane, for example the cell membrane of a eukaryotic cell. The transmembrane domain serves to transmit activation signals to the cytoplasmic signal transduction domains following ligand binding of the extracellular antigen-binding domains (e.g. scFv). The transmembrane domain of a CAR of the invention is typically the transmembrane domain of CD28. The transmembrane domain may be a transmembrane domain of the α, β, δ or γ subunits of the T-cell receptor, CD3ε, CD35, CD4, CD6, CD8a, CD28, CD86, OX-40, 4-1BB or CD40L (CD154). The transmembrane domain may be the transmembrane domain of CD8, for example, when the immune effector cell is an NK cell.

The intracellular signalling domain of a CAR of the invention may comprise any activation domain in the art. The activation domain serves to activate the immune effector cell following engagement of the extracellular domain (e.g. scFv). The intracellular signalling domain of a CAR of the invention may comprise one or more of a CD3ζ (zeta) activation domain, a 4-1BB (CD137) activation domain, a CD3ε (epsilon) activation domain, an OX40 (CD134) activation domain, a CD28 activation domain, and/or a CD27 activation domain.

The intracellular signalling domain may comprise a CD3ζ (zeta) activation domain, e.g. as in first generation CARs in the art.

The intracellular signalling domain may comprise a CD3ζ (zeta) activation domain and a CD28 activation domain, which may also be referred to as a CD28z domain, e.g. as in second generation CARs in the art.

The intracellular signalling domain may comprise a 4-1BB activation domain and a CD3ζ (zeta) activation domain, which may also be referred to as a 4-1BBz domain, e.g. as in third generation CARs in the art.

The intracellular signalling domain may comprise a 4-1BBz and a CD28z domain, which comprises CD3zeta, CD28 and the 4-1BB activation domains.

The intracellular signalling domain may comprise the CD3ζ (zeta) activation domain alone or in combination with a CD28, CD27, OX-40 (CD134) and/or 4-1BB (CD137) domain.

Typically, the intracellular signalling domain does not comprise a CD2 domain, such as a CD2 intracellular signalling domain.

Other activation domains include IL-15Ra, CD2, CDS, ICAM-1, LTA-1 and ICOS and may be used in combination with the activation domains described above.

If the immune effector cell in which the CAR is expressed is a phagocyte, the intracellular signalling domain may comprise the intracellular domain of Megf10 or FcRv.

A CAR of the invention may be expressed on the surface of the immune effector cell independently of the endogenous TCR-CD3 complex. The CAR of the invention may not require endogenous TCR and/or CD3 for expression on the surface of the immune effector cell.

### Accessory Receptors

An immune effector cell of the invention comprises an accessory receptor, which co-localises with a CAR on the cell membrane at an immunological synapse with an antigen presenting cell (APC). An accessory receptor is capable of binding to a ligand on an antigen presenting cell, and comprises an extracellular ligand binding domain, a transmembrane domain and an intracellular signalling domain.

An immune effector cell of the invention may comprise an accessory receptor of the invention, which is a modified accessory receptor. In this embodiment, the immune effector cell may also comprise a CAR of the invention.

An accessory receptor of the invention provides improved antigen sensitivity of the CARs expressed on the same immune effector cell compared to the corresponding endogenous accessory receptor. The accessory receptor of the invention is of a size such that the intermembrane distance spanned by the accessory receptor-ligand complex is comparable with the intermembrane distance spanned by the CAR-antigen complex. Hence, the size (e.g. height) of the extracellular portion of an accessory receptor, in particular, the height of the extracellular ligand binding domain of the accessory receptor from the membrane of the immune effector cell, is important in determining antigen sensitivity of a CAR expressed on the same immune effector cell. The optimal size (e.g. height) of the extracellular portion of an accessory receptor, in particular, the optimal height of the extracellular ligand binding domain of the accessory receptor from the membrane of the immune effector cell, is dependent on the sizes (e.g. heights) of its ligand, the CAR and the target antigen, as explained further below.

An accessory receptor of the invention may comprise an extracellular ligand binding domain having a height that is taller than the extracellular ligand binding domain of the corresponding endogenous accessory receptor from the membrane of an immune effector cell, e.g. by ≤15%, ≤20%, ≤25%, or ≤30%.

The extracellular ligand binding domain of an accessory receptor of the invention may have a height taller than about 7 nm but shorter than about 47 nm from the membrane of an immune effector cell. The extracellular ligand binding domain of an accessory receptor of the invention may have a height of >7 nm, ≥10 nm, ≥15 nm, ≥20 nm, ≥25 nm, ≥30 nm, ≥35 nm, ≥40 nm, ≥45 nm from the membrane of an immune effector cell. The extracellular ligand binding domain of an accessory receptor of the invention may have a height of ≤45 nm, ≤40 nm, ≤35 nm, ≤30 nm, ≤25 nm, ≤20 nm, ≤15 nm, or ≤10 nm from the membrane of an immune effector cell.

An accessory receptor of the invention may comprise an extracellular ligand binding domain that is shorter than the extracellular ligand binding domain of the corresponding endogenous accessory receptor from the membrane of an immune effector cell, e.g. by ≤15%, ≤20%, ≤25%, or ≤30%.

The extracellular ligand binding domain of an accessory receptor of the invention may have a height taller than about 3 nm but shorter than about 7 nm from the membrane of an immune effector cell. The extracellular ligand binding domain of an accessory receptor of the invention may have a height of ≥3 nm, ≥4 nm, ≥5 nm or ≥6 nm from the membrane of an immune effector cell. The extracellular ligand binding domain of an accessory receptor of the invention may have a height of <7 nm, ≤6 nm, ≤5 nm, or ≤4 nm from the membrane of an immune effector cell.

The height of the extracellular ligand binding domain of an accessory receptor of the invention from the membrane of an immune effector cell may be determined according to routine methods in the art, as described herein.

The stalk between the ligand binding domain and the transmembrane domain may determine the size (e.g. height) of the extracellular portion, in particular, the height of the extracellular ligand binding domain, of an accessory receptor of the invention. The inventors found that conventional CARs have poor antigen sensitivity. This may be improved by modulating (e.g. increasing or reducing) the size of the stalk of the accessory receptor (e.g. CD2), allowing optimal membrane alignment of the conventional CAR.

Hence, an accessory receptor of the invention comprises a stalk of the different size compared to the corresponding endogenous accessory receptor, such that the CAR of the invention may have a smaller or larger extracellular portion, and as a consequence a shorter or taller extracellular ligand binding domain than the corresponding endogenous accessory receptor.

The stalk of an accessory receptor of the invention comprises or consists of a sequence that physically increases the height of the ligand binding domain from the membrane of the immune effector cell.

The sequence may be an inflexible sequence, e.g, wherein each block of residues of the sequence physically increases the height of the ligand binding domain from the membrane of the immune effector cell because the sequence does not compress or fold back on itself. The block may comprise one, two, three, four, five, six, seven, eight, nine, ten or more residues.

The sequence may be a bulky sequence that physically increases the height of the ligand binding domain from the membrane of the immune effector cell, e.g. through steric hindrance, and thus prevents the ligand binding domain from contacting the membrane. The sequence that physically increases the height of the ligand binding domain from the membrane of the immune effector cell may be as described above for the hinge of the CAR of the invention.

The sequence that physically increases the height of the ligand binding domain from the membrane of the immune effector cell may be a mucin-like sequence, or fragments or derivatives thereof. Mucin like sequences are characterised by being rich in protein, serine and threonine residues, and the serine and threonine residues are heavily O-glycosylated. The mucin-like sequence may be a mucin-like sequence of the extracellular portion of CD43, as set out in SEQ ID 62. Mucin-like sequences from other proteins may also be used, such as MUC1, MUC3A, MUC3B, MUC4, MUC12, MUC13, MUC15, MUC16, MUC17, MUC18, MUC20, MUC21, PSGL-1. Stalks of surface proteins that are mucin-like, such as CD8a and CD28, may also be used.

The mucin-like sequence may be a fragment of the extracellular domain of CD43, as set out in SEQ ID 62. For example, the fragment may be between 4 and 234 amino acids (e.g. as shown in FIG. 1E). The fragment may consist of ≥5, ≥10, ≥20, ≥30, ≥40, ≥50, ≥50, ≥70, ≥80, ≥90, ≥100, ≥110, ≥120, ≥130, ≥140, ≥150, ≥160, ≥170, ≥180, ≥190, ≥200, ≥210, ≥220, ≥230 contiguous amino acids from SEQ ID NO: 62, e.g. from the N-terminus end or the C-terminus end of SEQ ID NO: 62. The fragment may consist of no more than 234 amino acids. The fragment may be between 4 and 120 amino acids, 8 and 80 amino acids, or 20 and 40 amino acids. The fragment may be any of SEQ ID NOs: 53 to 62.

In some cases, the sequence that physically increases the height of the ligand binding domain from the membrane of the immune effector cell is not an immunoglobulin domain.

The accessory receptor may be any of the receptors listed in Table 1. Thus, the ligand may be CD2, L-selectin, an α4 integrin, LFA-1, CD28, PD-1, 4-1BB or CD6. The accessory receptor may be an adhesion receptor. For example, the accessory receptor may be CD2.

TABLE 1
Examples of accessory receptors and its ligand

| Accessory receptor | Ligand |
|---|---|
| CD2 (also known as LFA-2) | CD58 or lymphocyte function-associated |
| (SEQ ID NO: 26) | antigen-1 (LFA-1) |
| L-selectin | glycosylation-dependent cell adhesion |
| | molecule-1 (GLYCAM1) |
| α4 integrins | vascular cell adhesion molecule-1 |
| | (VCAM-1) |
| lymphocyte | inter-cellular adhesion molecule 1 |
| function-associated | (ICAM-1, 2 & 3) |
| antigen-1 (LFA-1) | |
| CD28 | CD80 and CD86 |
| PD-1 | PD-L1 and PD-L2 |
| 4-1BB | 4-1BBL |
| CD6 | CD166 (ALCAM) |

Thus, the ligand may be CD58, GLYCAM1, VCAM-1, ICAM-1, ICAM-2, ICAM-3, CD80, CD86, PD-L1, PD-L2, 4-1BBL or CD166. The ligand may be CD58. Typically, the ligand on the APC is different to the antigen on the APC.

The accessory receptor may comprise the extracellular ligand binding domain of CD2, and the ligand may be CD58. The accessory receptor may be a modified CD2 according to the invention, and the ligand may be CD58.

### Membrane Alignment and Intermembrane Distance

Membrane alignment is influenced by the dimensions of the receptors and ligand complexes, such as CARs and accessory receptors that co-localise on the immune effector cell and their respective antigens and ligands on the APC. Membrane alignment for antigen recognition by CARs is optimised when the intermembrane distance spanned by the complex between the CARs and the target antigen is comparable with the intermembrane distance spanned by the complex between certain accessory receptors and their ligands.

The intermembrane distance spanned by the complex between the CAR and the target antigen is comparable with (e.g. within ≤5%, ≤10%, ≤15%, ≤20%, ≤25%, or ≤30%, or is the same as) the intermembrane distance spanned by the complex between the accessory receptor and the ligand.

The intermembrane distance spanned by the complex between a CAR of the invention and the target antigen may be comparable with (e.g. within ≤5%, ≤10%, ≤15%, ≤20%, ≤25%, or ≤30%, or is the same as) the intermembrane distance spanned by the complex between a T cell receptor and its peptide-MHC antigen.

The intermembrane distance spanned by the complex between the CAR of the invention and its antigen may be about 6 nm and 14 nm. The intermembrane distance spanned by the complex between the CAR of the invention and its antigen may be ≥6 nm, ≥7 nm, ≥8 nm, ≥9 nm, ≥10 nm, ≥11 nm, ≥12 nm or ≥13 nm. The intermembrane distance spanned by the complex between the CAR of the invention and its antigen may be ≤14 nm, ≤13 nm, ≤12 nm, ≤11 nm, ≤10 nm, ≤9 nm, ≤8 nm or ≤7 nm.

The intermembrane distance spanned by the complex between the CAR of the invention and its antigen may be about 14 nm and 54 nm. The intermembrane distance spanned by the complex between the CAR of the invention and its antigen may be ≥15 nm, ≥20 nm, ≥25 nm, ≥30 nm, ≥35 nm, ≥40 nm, ≥45 nm or ≥50 nm. The intermembrane distance spanned by the complex between the CAR of the invention and its antigen may be ≤50 nm, ≤45 nm, ≤40 nm, ≤35 nm, ≤30 nm, ≤25 nm, ≤20 nm or ≤15 nm.

Similarly, the intermembrane distance spanned by the complex between the accessory receptor of the invention and its ligand may be about 14 nm and 54 nm. The intermembrane distance spanned by the complex between the accessory receptor of the invention and its ligand may be ≥15 nm, ≥20 nm, ≥25 nm, ≥30 nm, ≥35 nm, ≥40 nm, ≥45 nm or ≥50 nm. The intermembrane distance spanned by the complex between the accessory receptor of the invention and its ligand may be ≤50 nm, ≤45 nm, ≤40 nm, ≤35 nm, ≤30 nm, ≤25 nm, ≤20 nm or ≤15 nm.

In an embodiment, the immune effector cell of the invention comprises a CAR of the invention (e.g. FIG. 1B), and the intermembrane distance spanned by the complex between the CAR of the invention and the target antigen is comparable with (e.g. within ≤5%, ≤10%, ≤15%, ≤20%, ≤25%, or ≤30%, or is the same as) the intermembrane distance spanned by the complex between the endogenous accessory receptor and its ligand. A CAR of the invention having an extracellular antigen binding domain of an appropriate height (h_{CAR}) may be determined based on the intermembrane distance spanned by the complex between the endogenous accessory receptor and its ligand (x), and the size (e.g. height) of the epitope of the antigen targeted by the CAR from the membrane of the APC (hₐₙₜ). The h_{CAR} may be comparable with (e.g. within ≤5%, ≤10%, ≤15%, ≤20%, ≤25%, or ≤30%, or is the same as) a value determined by h_{CAR}=x−hₐₙₜ. The h_{CAR} may be determined by x−hₐₙₜ. Examples of a CAR of the invention having an extracellular antigen binding domain of an appropriate height from the membrane of an immune effector cell are provided herein.

In the embodiment where the immune effector cell of the invention comprises a CAR_{CON} (i.e. a CAR comprising a CD28 hinge or a CD8a hinge) and an accessory receptor of the invention (e.g. FIG. 1C), the intermembrane distance spanned by the complex between the CAR_{CON} and the target antigen is comparable with (e.g. within ≤5%, ≤10%, ≤15%, ≤20%, ≤25%, or ≤30%, or is the same as) the intermembrane distance spanned by the complex between the accessory receptor of the invention and its ligand. An accessory receptor of the invention having an extracellular ligand binding domain of an appropriate height (h_{acc}) from the membrane of the immune effector cell may be determined by the intermembrane distance spanned by the complex between the CAR_{CON} and the target antigen (y), and the height of the epitope of the ligand (h_{lig}). The h_{acc} may be comparable with (e.g. within ≤5%, ≤10%, ≤15%, ≤20%, ≤25%, or ≤30%, or is the same as) a value determined by h_{acc}=y−h_{lig}. The h_{acc} may be determined by h_{acc}=y−h_{lig}. Examples of an accessory receptor of the invention having an extracellular ligand binding domain of an appropriate height from the membrane of an immune effector cell are provided herein.

In the embodiment where the immune effector cell comprises a CAR of the invention and an accessory receptor of the invention (e.g. FIG. 1D), the intermembrane distance spanned formed by the complex between the CAR of the invention and the target antigen is comparable with (e.g. within ≤5%, ≤10%, ≤15%, ≤20%, ≤25%, or ≤30%, or is the same as) the intermembrane distance spanned by the complex between the accessory receptor of the invention and its ligand. A CAR of the invention having an extracellular antigen binding domain of an appropriate height (h_{CAR}) and an accessory receptor of the invention having an extracellular ligand binding domain of an appropriate height (h_{acc}) from the membrane of the immune effector cell may be determined by considering the height of the epitope of the antigen (hₐₙₜ) and the ligand (h_{lig}), e.g. by h_{CAR}+hₐₙₜ=h_{acc}+h_{lig}. Examples of a CAR of the invention having an extracellular antigen binding domain of an appropriate height (h_{CAR}) and an accessory receptor of the invention having an extracellular ligand binding domain of an appropriate height from the membrane of the immune effector cell are provided herein.

Hence, the invention also relates to a method of determining an appropriate size (e.g. height) of the extracellular antigen binding domain of a CAR of the invention, and/or an appropriate size (e.g. height) of the extracellular ligand binding domain of an accessory receptor of the invention.

The intermembrane distance, height of the extracellular antigen binding domain or extracellular ligand binding domain from the membrane of an immune effector cell of any of the membrane proteins described herein may be determined according to routine methods in the art. For example, (1) using theoretical methods, e.g. as used herein; (2) using existing structural information to predict the overall size, such as from data in the protein data bank, (3) bioinformatics prediction tools based on the amino acid sequence of a protein, such as using Alphafold where sequences are converted into structures from which distances can be estimated, or (4) microscopy such as electron microscopy (7), immunofluorescence or quantum dot labelling. For example, the intermembrane distance spanned by the CD2-CD58 complex has been determined in references 3 and 6. Theoretical methods, as used herein, comprise adding a size to the known height of the extracellular portion of a protein, such as adding 3.7 nm for each immunoglobulin domain and/or 0.2 nm for each inflexible amino acid added (based on experimental data from reference 8). Electron microscopy can be used to directly measure the inter membrane distance. For example, T cells are incubated with APCs presenting a high concentration of antigen to the TCR or CAR. Direct inter membrane distances are taken at several places at the contact interface (see references 7 and 9). Indirect methods may also be used, such as by comparing the activity a CAR of the invention with that of a TCR, e.g. see Examples below.

### Immune Effector Cells

An immune effector cell of the invention comprises a CAR and/or an accessory receptor of the invention. The immune cell expresses a CAR and/or an accessory receptor of the invention.

An immune effector cell of the invention may comprise a CAR of the invention. The CAR of the invention may co-localise with endogenous accessory receptors, e.g. endogenous CD2, on the immune effector cell surface at an immunological synapse (e.g. FIG. 1B).

An immune effector cell of the invention may comprise an accessory receptor of the invention. The accessory receptor of the invention may co-localise with a CAR_{CON} (i.e. having a CD28 hinge as set out in SEQ ID NO: 10 or a CD8a hinge as set out in SEQ ID NO: 43) on the immune effector cell surface at an immunological synapse (e.g. FIG. 1C).

An immune effector cell of the invention may comprise a CAR of the invention and an accessory receptor of the invention. The CAR of the invention may co-localise with the accessory receptor of the invention on the immune effector cell surface at an immunological synapse (e.g. FIG. 1D).

An immune effector cell of the invention may be a T cell, a γδ T cell, a natural killer (NK) cell, an NKT cell, an induced pluripotent stem cell (iPSC) derived NK cell (iPSC-NK), a phagocyte, or a macrophage.

The immune effector cell may be a T cell. The T cell may be a CD8+ T cell, or cytotoxic T cell. The T cell may be a CD4-CD8+ T cell.

The T cell may be a CD4+ T cell, or helper T cell (TH cell), such as a TH1, TH2, TH3, TH17, TH9, or TFH cells. The T cell may be a regulatory T cell (Treg). The T cell may be a naïve, effector, memory, effector memory, central memory, memory stem T cell. The T cell may be a peripheral lymphocyte.

The T cell may be expanded from PBMCs. The T cell may be autologous with respect to a subject into which it is to be administered. The T cell may be allogeneic with respect to a subject into which it is to be administered. The T cell may be partially HLA-mismatched with respect to a subject into which it is to be administered.

The NK cell may be a cell of the NK92 cell line. The NK cell may be isolated from plasma blood mononuclear cells (PBMCs) of the subject to be treated or of a healthy donor. The NK cell may be isolated from cord blood. The NK cell may be differentiated from a CD34⁺ haematopoietic progenitor cell (HPC).

The macrophage may be differentiated into the “M1” phenotype. The M1 macrophage expresses pro-inflammatory cytokines and has strong anti-tumour activity. An undifferentiated macrophage expressing a CAR described herein may be induced to differentiate into the M1 phenotype by culturing in the presence of the antigen.

Sources of cells for use in accordance with the invention will be known to persons skilled in the art, illustrative examples of which include peripheral blood, peripheral blood mononuclear cells, bone marrow, lymph nodes tissue, cord blood, thymus tissue, tissue from the site of infection, ascites, pleural effusion, spleen tissue, and tumors. In an embodiment, the cells are derived from whole blood.

An immune effector cell of the invention may be derived from an autologous cell. An immune effector cell may be derived from an allogeneic cell. The term “autologous” refers to any material derived from the same individual to whom the material is later to be re-introduced to the individual. The term “allogeneic” refers to any material derived from a different individual of the same species as the individual to whom the material is introduced. Two or more individuals are said to be allogeneic to one another when the genes at one or more loci are not identical. In some aspects, allogeneic materials from individuals of the same species may be sufficiently genetically distinct to interact antigenically.

The immune effector cell may comprise a nucleic acid described herein. The immune effector cell may comprise a vector described herein. The immune effector cell may comprise an RNA or RNA vector described herein.

The immune effector cell may express a CAR specific for one or more antigens.

The immune effector cell may permanently or transiently express a CAR and/or an accessory protein of the invention.

The immune effector cell may comprise a modification in its genome to reduce or eliminate the expression of the endogenous accessory receptor. The endogenous accessory receptor may be any of the accessory receptor described herein, e.g. in Table 1. The endogenous accessory receptor may be an adhesion receptor, such as CD2. Genetic modification techniques for such modifications are well known in the art.

The invention also provides an immune effector cell obtained or obtainable by any of the methods described herein.

The invention also relates to a method of preparing an immune effector cell of the invention or a population of immune effector cells of the invention. The method comprises introducing a nucleic acid encoding a CAR and/or an accessory receptor of the invention into an immune effector cell, e.g. by transformation (such as transfection or transduction).

The term “transduction” may be used to describe virus mediated nucleic acid transfer. A viral vector may be used to transduce the cell with the one or more constructs. Conventional viral based expression systems could include retroviral, alpha-retroviral, lentivirus, adenoviral, adeno-associated (AAV) and herpes simplex virus (HSV) vectors for gene transfer. Non-viral transduction vectors include transposon-based systems including PiggyBac and Sleeping Beauty systems. Methods for producing and purifying such vectors are known in the art. The vector is preferably a vector described herein. Immune effector cells may be transduced using any method known in the art. Transduction may be in vitro or ex vivo.

The term “transfection” may be used to describe non-virus-mediated nucleic acid transfer. The immune effector cells may be transfected using any method known in the art. Transfection may be in vitro or ex vivo. Any vector capable of transfecting immune effector cells may be used, such as conventional plasmid DNA or RNA transfection, preferably mRNA transfection. A human artificial chromosome and/or naked RNA may be used to transfect the cell with the nucleic acid sequence or nucleic acid construct. Human artificial chromosomes are described in e.g. Kazuki et al., Mol. Ther. 19(9): 1591-1601 (2011), and Kouprina et al., Expert Opinion on Drug Delivery 11(4): 517-535 (2014). Alternative non-viral delivery systems include DNA plasmids, naked nucleic acid, and nucleic acid complexed with a delivery vehicle, such as a liposome. Methods of non-viral delivery of nucleic acids include lipofection, microinjection, biolistics, virosomes, liposomes, immunoliposomes, polycation or lipid: nucleic acid conjugates, naked DNA, naked RNA, artificial virions, and agent-enhanced uptake of DNA.

Nanoparticle delivery systems may be used to transfect the immune effector cell with the nucleic acid sequence. Such delivery systems include, but are not limited to, lipid-based systems, liposomes, micelles, microvesicles and exosomes. With regard to nanoparticles that can deliver RNA, see, e.g., Alabi et al., Proc Natl Acad Sci USA. 2013 Aug. 6; 110(32):12881-6; Zhang et al., Adv Mater. 2013 Sep. 6; 25(33):4641-5; Jiang et al., Nano Lett. 2013 Mar. 13; 13(3):1059-64; Karagiannis et al., ACS Nano. 2012 Oct. 23; 6(10):8484-7; Whitehead et al., ACS Nano. 2012 Aug. 28; 6(8):6922-9 and Lee et al., Nat Nanotechnol. 2012 Jun. 3; 7(6):389-93. Lipid Nanoparticles, Spherical Nucleic Acid (SNA™) constructs, nanoplexes and other nanoparticles (particularly gold nanoparticles) are also contemplated as a means for delivery of a nucleic acid or vector of the invention.

The immune effector cell may be transfected by electroporation. The electroporation is mRNA electroporation. This has the advantage of allowing transient expression of the CAR and/or accessory receptor.

The invention also relates to a method, such as an ex vivo method, of preparing a population of immune effector cells (e.g. for adoptive cell therapy), comprising culturing the immune effector cell of the invention to produce a population of immune effector cells.

The invention also provides a population of immune effector cells obtained or obtainable by any of the methods described herein. The invention also provides a population of immune effector cells that express a CAR and/or an accessory receptor of the invention.

The immune effector cell induces an effector function on the antigen presenting cell that it contacts. The effector function may be immune effector cell activation (e.g. T cell activation), immune effector cell proliferation, (e.g. T cell proliferation) cytolytic activity (e.g. apoptosis or cell death of the APC), the arrest or reduction of cell growth (e.g. of the APC), and/or modulating cytokine release (such as release of pro-inflammatory cytokines). Methods of measuring the effector function are well known in the art. The immune effector cell activation may be measured by increased surface expression of 4-1BB and/or CD25, and/or increased expression of CD69. The cytolytic activity may be measured by cell death of target cells, and/or release of perforin and/or granzyme. The modulation of cytokine release may be an increase in the release of pro-inflammatory cytokines. The pro-inflammatory cytokines may be selected from GM-CSF, TNF-α, IL-2, IL-6, IL-1β and/or IFN-γ.

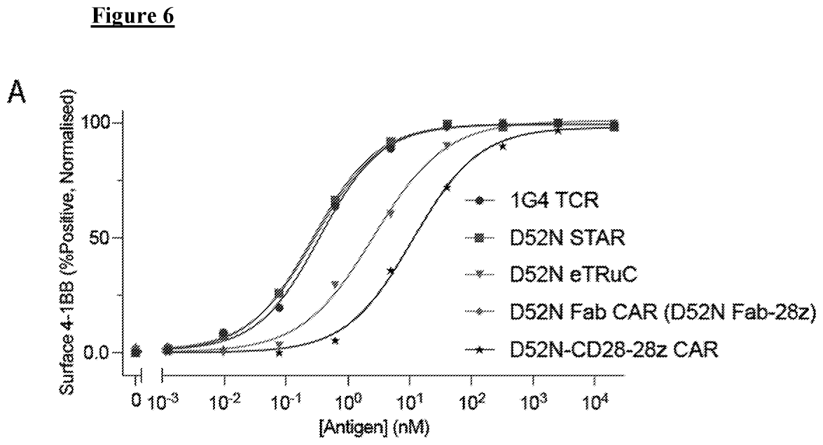

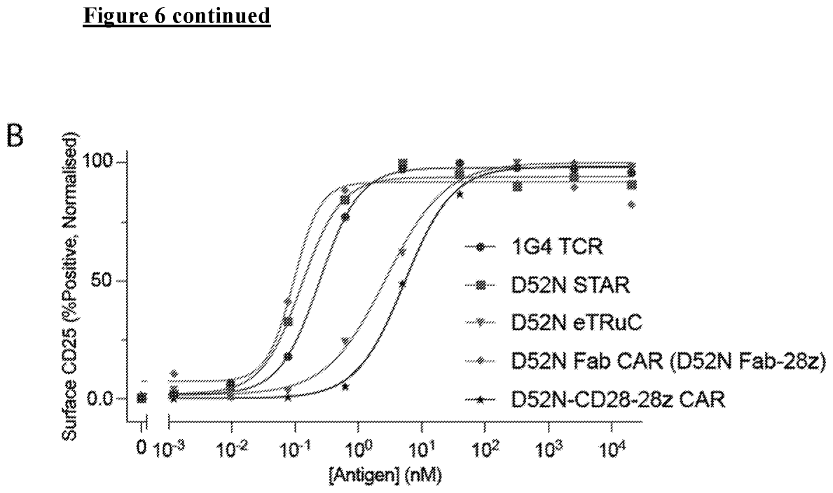

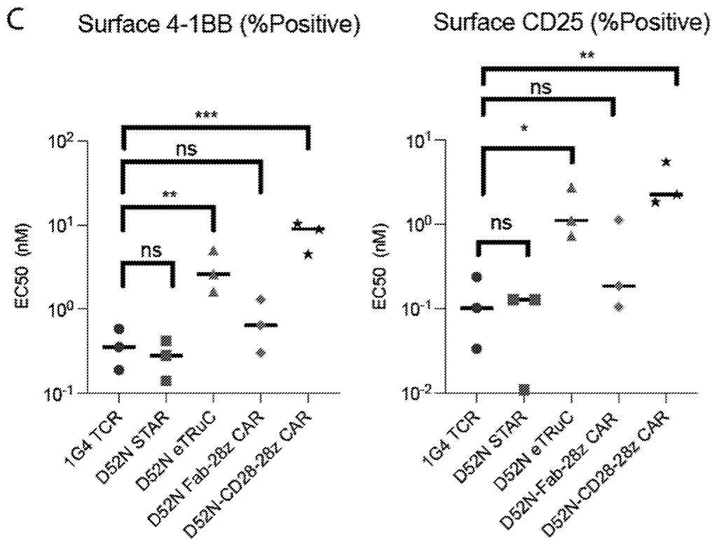

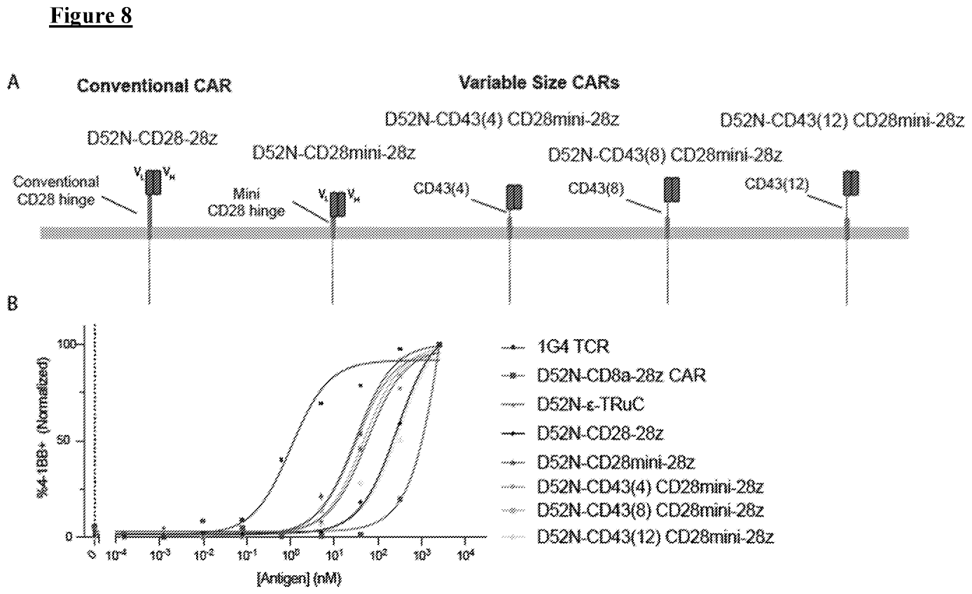

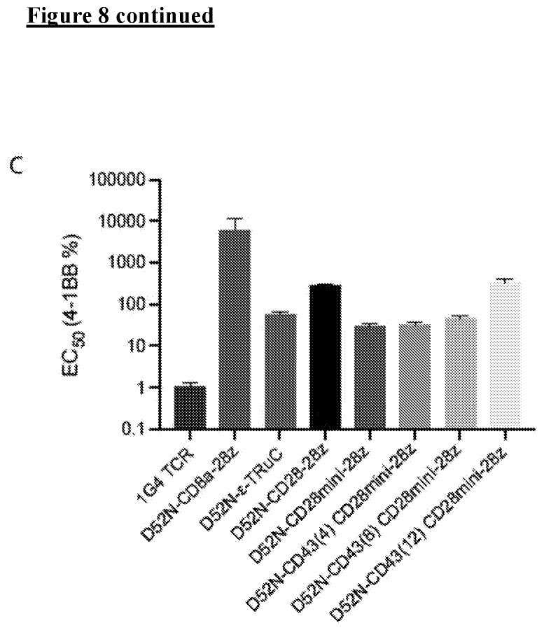

Surface expression of 4-1-BB and/or CD25 may be measured as a normalised percentage of maximal surface expression, as shown in FIGS. 6 and 8.

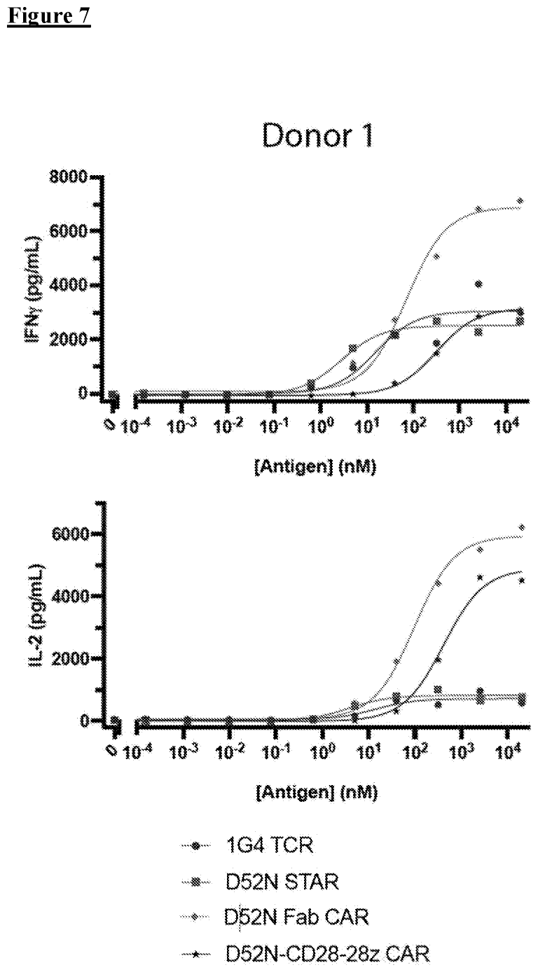

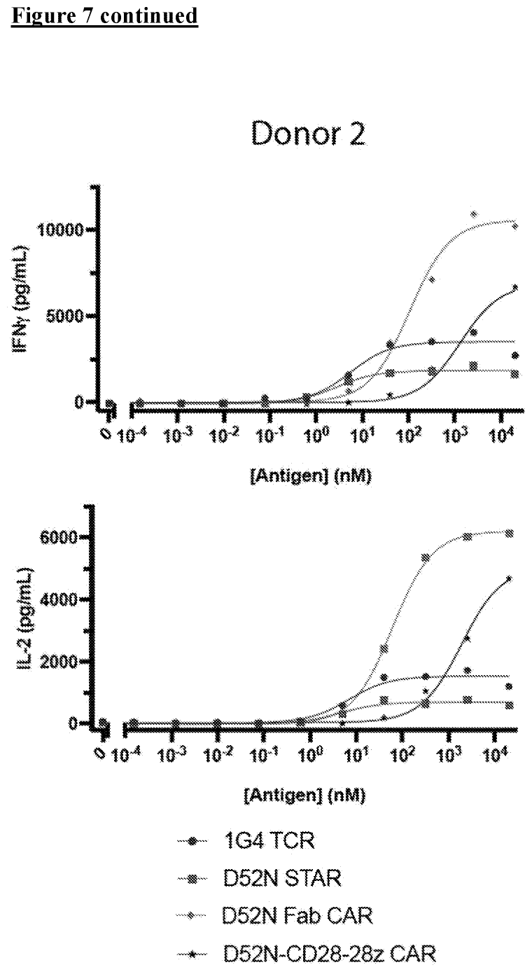

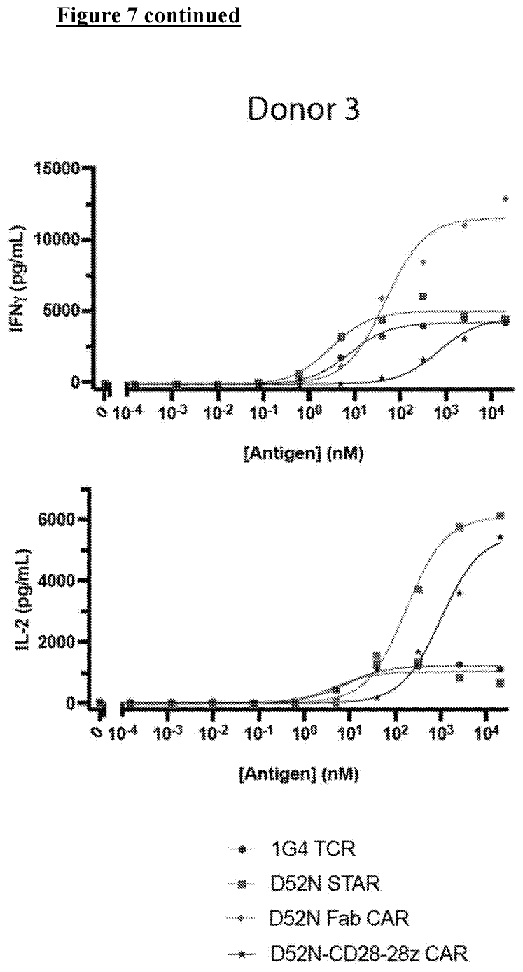

The release of pro-inflammatory cytokines may be measured as shown in FIG. 7. Preferably, the cytokines IL-2 and/or IFN-γ are measured.

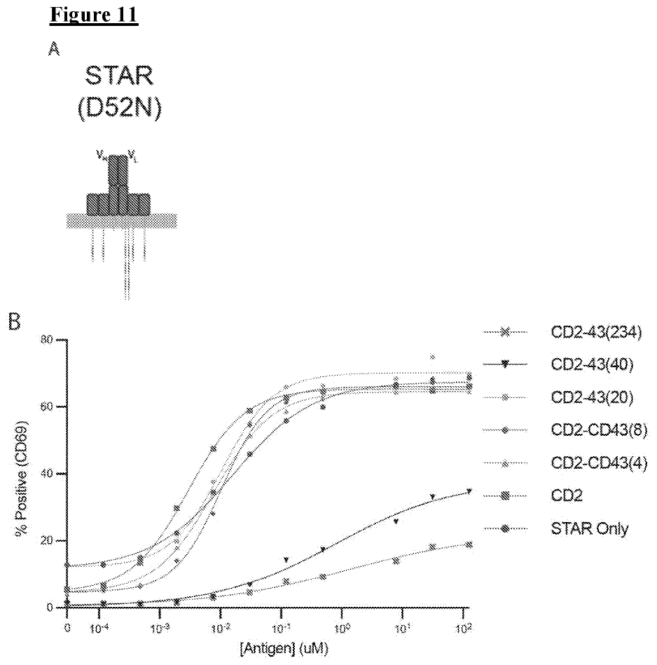

Expression of CD69 may be measured as a percentage of cells positive for CD69, as shown in FIGS. 10 to 14.

The immune effector cell expressing the CAR of the invention and/or the accessory receptor of the invention may have improved or optimised effector function when compared to an immune effector cell expressing a conventional CAR comprising the same antigen-binding domain. The effector function is improved when immune effector cell activation is increased, immune effector cell proliferation is increased, cytolytic activity is increased, the proliferation of the APC is slowed or halted, the release of pro-inflammatory cytokines is increased and/or the release of anti-inflammatory cytokines is decreased. The effector function may be measured as an EC50 for comparative purposes, as shown in the Figures. The effector function may be increased by 10% or more, 15% or more, 20% or more, 25% or more, 30% or more, 35% or more, 40% or more, 45% or more, 50% or more, 60% or more, 70% or more, 80% or more, 90% or more, 100% or more, or 200% or more. The effector function is optimal when varying the size of the CAR and/or the accessory receptor does not lead to further improvements in the effector function. The effector function may be considered optimised when it is within +/−25% of the optimal effector function, such as +/−20%, +/−15%, +/−10%, or +/−5% of the optimal effector function.

### Antigen Presenting Cell

Any antigen presenting cell (APC) is useful with the invention. The APC may be a professional APC, such as a dendritic cell, a macrophage, B-cell, or an epithelial cell. The APC may be a non-professional APC, such as a fibroblast, a thymic epithelial cell, a thyroid epithelial cell, a glial cell, a pancreatic beta cell, or a vascular endothelial cell. The APC may be a tumour cell, e.g. a tumour cell of the tumours provided in Table 2.

The APC comprises an antigen on its cell surface that is capable of binding to the antigen binding domain of a CAR, such as a CAR of the invention. The antigen may be any antigen, such as a tumour antigen (e.g. a tumour associated antigen, a development tumour antigen, and/or a neo-antigen), e.g. as listed in Table 2. The antigen may be a peptide-MHC complex, CD19, mesothelin, BCMA, CD22, EGFR, EGFRVIII, or NY-ESO-1.

TABLE 2
Examples of antigens targeted by
CARs and associated malignancies

| Antigen | Associated Malignancy |
|---|---|
| α-Folate receptor | Ovarian cancer, epithelial cancers |
| CAIX | Renal-cell carcinoma |
| CD19 | B cell malignancies, Refractory Follicular, |
| | Lymphoma, ALL, CLL |
| CD20 | Lymphomas, B cell malignancies, B-cell |
| | lymphomas, Mantle cell lymphoma, |
| | indolent B cell lymphomas |
| CD22 | B cell malignancies |
| CD23 | CLL |
| CD24 | Pancreatic adenocarcinoma |
| CD30 | Lymphomas, Hodgkin lymphoma |
| CD33 | AML |
| CD38 | Non Hodgkin lymphoma |
| CD44v7/8 | Cervical carcinoma |
| CEA | Colorectal cancer |
| EGFRvIII | Glioblastoma |
| EGP-2 | Multiple malignancies |
| EGP-40 | Colorectal cancer |
| EphA2 | Glioblastoma |
| erb-B2 | Breast cancer, Prostate cancer, |
| | Colon cancer, Various tumors |
| erb-B 2, 3, 4 | Breast cancer |
| FBP | Ovarian cancer |
| Fetal acetylcholine | Rhabdomyosarcoma |
| receptor | |
| G_{D2} | Neuroblastoma, Melanoma, Ewing sarcoma |
| G_{D3} | Melanoma |
| Her-2 | Medulloblastoma, Pancreatic |
| | adenocarcinoma, Glioblastoma, |
| | Osteosarcoma, Ovarian cancer |
| HMW-MAA | Melanoma |
| IL-11Rα | Osteosarcoma |
| IL-13R-α2 | Glioma, Glioblastoma, Medulloblastoma |
| KDR | Tumor neovasculature |
| κ-light chain | B-cell malignancies (B-NHL, CLL) |
| Lewis Y | Various carcinomas, Epithelial |
| | derived tumors |
| L1-cell adhesion | Neuroblastoma |
| molecule | |
| MAGE-A1 | Melanoma |
| Mesothelin | Mesothelioma |
| Murine CMV | Murine CMV |
| infected cells | |
| MUC1 | Breast, Ovary |
| MUC16 | Ovary |
| NKG2D Ligands | Myeloma, ovarian & other tumors |
| NY-ESO-1 | Multiple myeloma |
| (157-165) | |
| Oncofetal antigen | Various tumors |
| (h5T4) | |
| PSCA | Prostate carcinoma |
| scFv-CD3ζ | |
| PSMA | Prostate cancer/tumor vasculature |
| Prostate/tumor | scFv-CD28-CD3ζ |
| vasculature | |
| ROR1 | B-CLL and mantle cell lymphoma |
| Targeting via mAb | Various tumors |
| IgE | |
| TAG-72 | Adenocarcinomas |
| VEGF-R2 | Tumor neovasculature |
| Biotinylated | Various tumors-ovarian |
| molecules | |

### Nucleic Acids, Vectors and Host Cells

Also provided is one or more isolated nucleic acids encoding a CAR and/or an accessory receptor of the invention. In some cases, the nucleic acid is collectively present on more than one nucleic acid, but collectively together they are able to encode a CAR and/or an accessory receptor of the invention.

Nucleic acids which encode a CAR and/or an accessory receptor of the invention can be obtained by methods well known to those skilled in the art. For example, DNA sequences coding for part or all of the antibody heavy and light chains may be synthesised as desired from the corresponding amino acid sequences.

The nucleic acid may be a DNA sequence. The nucleic acid may be an RNA sequence, such as mRNA. A vector may comprise the nucleic acid.

The vector may be a viral vector. Conventional viral based expression systems could include retroviral, alpha-retroviral, lentivirus, adenoviral, adeno-associated (AAV) and herpes simplex virus (HSV) vectors for gene transfer. Non-viral transduction vectors include transposon-based systems including PiggyBac and Sleeping Beauty systems. Methods for producing and purifying such vectors are known in the art.

The vector may be a cloning vector or an expression vector. A suitable vector may be any vector which is capable of carrying a sufficient amount of genetic information, and allowing expression of a polypeptide of the invention.

The vector is preferably an RNA vector. Suitable RNA vectors include the RNA vectors as described in Schutsky, Keith, et al., Oncotarget 6.30 (2015): 28911 and Beatty, Gregory L., et al., Gastroenterology 155.1 (2018): 29-32.

General methods by which the vectors may be constructed, transfection methods and culture methods are well known to those skilled in the art. In this respect, reference is made to “Current Protocols in Molecular Biology”, 1999, F. M. Ausubel (ed), Wiley Interscience, New York and the Maniatis Manual produced by Cold Spring Harbor Publishing.

A nucleic acid may be provided in the form of an expression cassette, which includes control sequences operably linked to the inserted sequence, thus allowing for expression of a CAR and/or an accessory receptor of the invention in vivo. Hence, also provided is one or more expression cassettes encoding the one or more nucleic acids that encode a CAR and/or an accessory receptor of the invention. These expression cassettes, in turn, are typically provided within vectors (e.g. plasmids or recombinant viral vectors). Hence, also provided is a vector encoding a CAR and/or an accessory receptor of the invention. Further provided are vectors which collectively encode a CAR and/or an accessory receptor of the invention.

The vector may be a human artificial chromosome. Human artificial chromosomes are described in e.g. Kazuki et al., Mol. Ther. 19(9): 1591-1601 (2011), and Kouprina et al., Expert Opinion on Drug Delivery 11(4): 517-535 (2014).

The vector may be a non-viral delivery system, such as DNA plasmids, naked nucleic acid (e.g. naked RNA), and nucleic acid complexed with a delivery vehicle, such as a liposome.

The nucleic acids, expression cassettes or vectors described herein may be introduced into a host cell, e.g. by transfection. Hence, also provided is a host cell comprising the one or more nucleic acids, expression cassettes or vectors of the invention. The nucleic acids, expression cassettes or vectors described herein may be introduced transiently or permanently into the host cell, allowing expression of an antibody from the one or more nucleic acids, expression cassettes or vectors. Such host cells include transient, or preferably stable higher eukaryotic cell lines, such as mammalian cells or insect cells, lower eukaryotic cells, such as yeast, or prokaryotic cells, such as bacteria cells. Particular examples of cells include mammalian HEK293, such as HEK293F, HEK293T, HEK293S or HEK Expi293F, CHO, HeLa, NSO and COS cells, or any other cell line used herein.

Typically, the host cell is an immune effector cell of the invention. The nucleic acids, expression cassettes or vectors described herein may be introduced transiently into the host cell.

The polynucleotide or vector of the invention may be an mRNA for administering to patients, e.g. mRNA vaccination. The patient's T cells may then express the CAR of the invention and/or the accessory receptor of the invention in vivo. Such mRNA molecules and associated methods are described in reference 10.

Also provided is a kit suitable for transforming and/or transfecting an immune effector cell or a population of immune effector cells to generate an immune effector cell or population of immune effector cells of the invention. The kit comprises a nucleic acid or vector described herein. The kit may comprise further agents such as those discussed herein that improve transfection or transformation efficacy.

### Methods

Also provided herein is a method of increasing and/or optimising the effector function of an immune effector cell, comprising modifying the size (e.g. height) of the extracellular portion of a CAR and/or the size (e.g. height) of the extracellular portion of an accessory receptor to optimise the effector function of the immune effector cell upon contact with the APC. In particular, the height of the extracellular antigen binding domain of the CAR and/or the extracellular ligand binding domain of the accessory receptor are modified. The invention also provides an immune effector cell obtained or obtainable by the method. The effector function may be any effector function as described herein.

Also provided herein a method for identifying an improved immune effector cell, comprising modifying the size (e.g. height) of the extracellular portion of a CAR and/or the size (e.g. height) of the extracellular portion of an accessory receptor, and hence the height of the extracellular antigen binding domain of the CAR and/or the extracellular ligand binding domain of the accessory receptor; and determining whether an immune effector cell expressing a modified CAR and/or a modified accessory receptor has improved effector function when compared to an immune effector cell expressing an unmodified CAR and/or unmodified accessory receptor. The invention also provides an immune effector cell obtained or obtainable by the method.

The CAR may be a CAR as described herein.

The accessory receptor may be an accessory receptor as described herein. The accessory receptor may be CD2.

A method of the invention may comprise modifying the size of the extracellular portion of the CAR and/or the size of the extracellular portion of the accessory receptor, and hence the height of the extracellular antigen binding domain of the CAR and/or the extracellular ligand binding domain of the accessory receptor, such that the intermembrane distance spanned by the complex between CAR and the antigen is comparable (e.g. within ≤5%, ≤10%, ≤15%, ≤20%, ≤25%, or ≤30%, or is the same as) with the intermembrane distance spanned by the complex between the accessory receptor and the ligand. The intermembrane distance spanned may be comparable with (e.g. within ≤5%, ≤10%, ≤15%, ≤20%, ≤25%, or ≤30%, or is the same as) the intermembrane distance spanned by the complex between a T cell receptor and the antigen.

A method of the invention may comprise reducing the size (e.g. height) of the extracellular portion of the CAR, and hence reducing the height of the extracellular antigen binding domain of the CAR.

A method of the invention may comprise increasing the size (e.g. height) of the extracellular portion of the CAR, and hence increasing the height of the extracellular antigen binding domain of the CAR from the membrane of an immune effector cell.

A method of the invention may comprise reducing the size (e.g. height) of the extracellular portion of the accessory receptor, and hence reducing the extracellular ligand binding domain of the accessory receptor from the membrane of an immune effector cell.

A method of the invention may comprise increasing the size (e.g. height) of the extracellular portion of the accessory receptor, and hence increasing the extracellular ligand binding domain of the accessory receptor from the membrane of an immune effector cell.

A method of the invention may comprise increasing the size (e.g. height) of the extracellular portion of the CAR and reducing the size (e.g. height) accessory receptor, and hence increasing the height of the extracellular antigen binding domain of the CAR from the membrane of an immune effector cell and reducing the height of the extracellular ligand binding domain of the accessory receptor from the membrane of an immune effector cell.

A method of the invention may comprise reducing the size (e.g. height) of the extracellular portion of the CAR and increasing the size (e.g. height) of the accessory receptor, and hence reducing the height of the extracellular antigen binding domain of the CAR from the membrane of an immune effector cell and the increasing the height of the extracellular ligand binding domain of the accessory receptor from the membrane of an immune effector cell.

Determination of the intermembrane distance spanned and appropriate size (e.g. height) of the extracellular portion of a CAR and/or an accessory receptor of the invention, e.g. the height of the extracellular antigen binding domain of the CAR and/or an accessory receptor of the invention from the membrane of an immune effector cell, is described herein.

A method of the invention may comprise introducing a sequence that physically increases the height of the antigen binding domain from the membrane of the immune effector cell in the extracellular portion in the CAR and/or accessory receptor, e.g. in the hinge of the CAR or the stalk of the accessory receptor. Suitable sequences that physically increases the height of the antigen binding domain from the membrane of the immune effector cell are described herein.

A method of the invention may comprise replacing the hinge of the CAR or the stalk of the accessory receptor with a sequence that physically increases the height of the antigen binding domain from the membrane of the immune effector cell. Suitable sequences that physically increases the height of the antigen binding domain from the membrane of the immune effector cell are described herein.

The invention also provides a method for identifying an improved immune effector cell, comprising determining whether an immune effector cell of the invention has improved effector function when compared to an immune effector cell expressing a corresponding unmodified CAR and/or a corresponding unmodified accessory receptor.

A method of the invention may further comprise determining the level of effector function of the immune effector cell, such as cell killing or cytokine production. Such methods are well known in the art, as described herein.

### Pharmaceutical Composition

Also provided is a composition comprising an immune effector cell or population of immune effector cells of the invention. The immune effector cell or population of immune effector cells may be at least 1% of the total cells in the composition, such as at least 5%, at least 10%, at least 15 at least 20%, at least 25%, at least 30%, at least 35%, at least 40%, at least 45%, at least 50%, at least 60%, at least 70%, at least 80%, at least 90%, at least 95%, at least 96%, at least 97%, at least 98%, at least 99% or at least 99.9% of the total cells in the composition. The total cells in the composition may consist or consist essentially of the immune effector cell or population of immune effector cells of the invention, i.e. no other cells are detectable in the composition.

The composition may be a pharmaceutical composition. The pharmaceutical composition may comprise a pharmaceutically acceptable carrier. Suitable pharmaceutically acceptable carriers comprise aqueous carriers, diluents or excipients. Examples of suitable carriers include all aqueous and non-aqueous isotonic sterile injection solutions which may contain anti-oxidants, buffers and solutes, which render the composition isotonic with the blood of the intended recipient; aqueous and non-aqueous sterile suspensions, which may include suspending agents and thickening agents, dispersion media, antifungal and antibacterial agents, isotonic and absorption agents and the like. It will be understood that compositions of the invention may also include other supplementary physiologically active agents.

The carrier is typically pharmaceutically “acceptable” in the sense of being compatible with the other ingredients in the composition and not injurious to the subject. Compositions include those suitable for parenteral administration, including subcutaneous, intramuscular, intravenous and intradermal administration. The compositions may conveniently be presented in unit dosage form and may be prepared by any method well known in the art of pharmacy. Such methods include preparing the carrier for association with the isolated T cells. In general, the compositions are prepared by uniformly and intimately bringing into association any active ingredients with liquid carriers.

The composition may be suitable for parenteral administration. In another embodiment, the composition is suitable for intravenous administration. Compositions suitable for parenteral administration include aqueous and non-aqueous isotonic sterile injection solutions which may contain anti-oxidants, buffers, bactericides and solutes, which render the composition isotonic with the blood of the intended recipient; and aqueous and non-aqueous sterile suspensions which may include suspending agents and thickening agents.

The composition described herein may be prepared in a manner known in the art and are those suitable for parenteral administration to mammals, particularly humans, comprising a therapeutically effective amount of the composition with one or more pharmaceutically acceptable carriers or diluents. The composition may comprise at least about 1×10⁶ to about 1×10¹² of the immune effector cells of the invention. The composition may comprise at least about 1×10⁷, at least about 1×10⁸, at least about 1×10⁹, at least about 1×10¹⁰ or at least about 1×10¹¹ of the immune effector cells of the invention.

The present disclosure also contemplates the combination of the composition described herein with other active agents and/or in addition to other treatment regimens or modalities such as radiation therapy or surgery. When the composition described herein is used in combination with known active agents, the combination may be administered either in sequence (either continuously or broken up by periods of no treatment) or concurrently or as an admixture.

Suitable anti-cancer agents will be known to persons skilled in the art.

Treatment in combination is also contemplated to encompass the treatment with either the composition of the invention followed by a known treatment, or treatment with a known agent followed by treatment with the composition of the invention, for example, as maintenance therapy.

For example, in the treatment of cancer it is contemplated that the composition of the present invention may be administered in combination with an alkylating agent (such as mechlorethamine, cyclophosphamide, chlorambucil, ifosfamidecysplatin, or platinum-containing alkylating agents such as cisplatin, carboplatin and oxaliplain), and anti-metabolite (such as a purine or pyrimidine analogue or an anti-folate agent, such as azathioprine and mercaptopurine), an anthracycline (such as daunorubicin, doxorubicin, epirubicin idarubicin, valrubicin, mitoxantrone or anthracycline analog), a plant alkaloid (such as a vinca alkaloid or a taxane, such as vincristine, vinblastine, vinorelbine, vindesine, paclitaxel or doestaxel), a topoisomerase inhibitor (such as a type I or type II topoisomerase inhibitor), a podophyllotoxin (such as etoposide or teniposide), a tyrosine kinase inhibitor (such as imatinib mesylate, nilotinib or dasatinib), an adenosine receptor inhibitor (such as A2aR inhibitors, SCH58261, CPI-444, SYN115, ZM241385, FSPTP or A2BR inhibitors such as PSB-1115), adenosine receptor agonists (such as CCPA, IB-MECA and CI-IB-MECA), a checkpoint inhibitor, including those of the PDL-1: PD-1 axis, nivolumab, pembrolizumab, atezolizumab, BMS-936559, MEDI4736, MPDL33280A or MSB0010718C), an inhibitor of the CTLA-4 pathway (such as ipilimumab and tremelimumab), an inhibitor of the TIM-3 pathway or an agonist monoclonal antibody that is known to promote T cell function (including anti-OX40, such as MEDI6469; and anti-4-BB, such as PF-05082566).

The invention also provides a kit or article of manufacture including a pharmaceutical composition as described above.

The invention also provides a kit for use in a therapeutic application mentioned above, the kit comprising:
(a) a container holding a polypeptide, nucleic acid, vector or pharmaceutical composition of the invention; and
(b) a label or package insert with instructions for use.

Suitable containers include, for example, bottles, vials, syringes, blister pack, etc. The containers may be formed from a variety of materials such as glass or plastic. The container holds a therapeutic composition which is effective for treating the condition and may have a sterile access port (e.g., the container may be an intravenous solution bag or a vial having a stopper pierceable by a hypodermic injection needle). The label or package insert indicates that the therapeutic composition is used for treating the condition of choice. In an embodiment, the label or package insert includes instructions for use and indicates that the therapeutic or prophylactic composition can be used to treat a cancer or other condition described herein.

The kit may further comprise a further container comprising a pharmaceutically-acceptable buffer, such as bacteriostatic water for injection (BWFI), phosphate-buffered saline, Ringer's solution and dextrose solution. It may further comprise other materials desirable from a commercial and user standpoint, which would be known to persons skilled in the art, suitable examples of which include other buffers, diluents, filters, needles, and syringes.

### Therapeutic Uses

Also described herein is use of a CAR, an accessory receptor, immune effector cell or population of immune effector cells of the invention, in a method of treatment of the human or animal body by therapy.

For instance, also provided is a method of treating cancer in a subject, the method comprising administering to the subject an effective amount of an immune effector cell or a population of immune effector cells of the invention. Hence, the invention also provides a CAR, an accessory receptor, an immune effector cell or a population of immune effector cells of the invention for use in a method of treating cancer. The invention also provides the use of a CAR, an accessory receptor, an immune effector cell or a population of immune effector cells of the invention for the manufacture of a medicament for the treatment of cancer. The invention also provides the use of a CAR, an accessory receptor, immune effector cell or population of immune effector cells of the invention to treat cancer.

Also provided is a method of performing adoptive cell therapy in a subject, the method comprising administering to the subject an effective amount of an immune effector cell or a population of immune effector cells of the invention. Hence, the invention also provides a CAR, an accessory receptor, an immune effector cell or a population of immune effector cells of the invention for use in adoptive cell therapy. The invention also provides the use of a CAR, an accessory receptor, an immune effector cell or a population of immune effector cells of the invention for the manufacture of a medicament for adoptive cell therapy. The invention also provides the use of a CAR, an accessory receptor, immune effector cell or population of immune effector cells of the invention adoptive cell therapy.

The cancer may be any cancer, such as a solid cancer. The cancer may be a malignancy listed in Table 2. The cancer may be haematological malignancy or B cell cancer.

The therapeutic uses and methods may comprise administering a therapeutically effective amount of the immune effector cell or population of immune effector cells.

Also provided is a method of formulating a composition for treating cancer, wherein said method comprises mixing an immune effector cell or population of immune effector cells of the invention with an acceptable carrier to prepare said composition.

The subject may have been previously treated for the cancer, such as using adoptive cell therapy.

The therapeutic methods and uses may comprise, prior to treatment with an immune effector cell or population of immune effector cells of the invention, determining whether the cancer expresses a target antigen specifically targeted by immune effector cell or population of immune effector cells of the invention.

The method may comprise selecting an immune effector cell or population of immune effector cells based on the expression of the target antigen by the cancer, so that the immune effector cell or population of immune effector cell is specific for the cancer. The method may comprise transfecting or transforming an immune effector cell with a nucleic acid of the invention in response to information on the expression of the target antigen by the cancer.

The therapeutic methods and uses described herein may comprise inhibiting the disease state (i.e. the cancer), for example by arresting its development and/or causing regression of the disease state until a desired end point is reached. The therapeutic methods and uses of the invention may comprise achieving a partial response, a full response by the cancer. The therapeutic methods and uses of the invention may achieve remission of the cancer.

The therapeutic methods and uses described herein may delay the growth of the cancer, arrest the growth of the cancer and/or reverse the growth of the cancer. The therapeutic methods and uses of the invention may reduce the size of the cancer by at least 10%, such as at least 20%, at least 30%, at least 40%, at least 50%, at least 60%, at least 70%, at least 80%, at least 90% or by 100%.

Typically, the therapeutic methods and uses are for a human subject in need thereof. However, non-human animals such as non-human mammals are also contemplated. The non-human mammals may be rats, rabbits, sheep, pigs, cows, cats or dogs.

The dose of the immune effector cell or population of immune effector cells may vary depending on the age and size of a subject, as well as on the disease, conditions and route of administration. The immune effector cell or population of immune effector cells may be administered at a dose of about 1×10⁶ to about 1×10¹² cells. The immune effector cell or population of immune effector cells may be administered at a dose of about 1×10⁵ cells/kg to about 1×10¹¹ cells/kg body weight.

The immune effector cell or population of immune effector cells may be administered as a single dose. The immune effector cell or population of immune effector cells may be administered in a multiple dose regimen. For example, the initial dose may be followed by administration of a second or plurality of subsequent doses. The second and subsequent doses may be separated by an appropriate time. For example, the doses between doses may be administered once about every week, once about every 2 weeks, once about every 3 weeks, once about every four weeks, or once about every month.

The immune effector cell or population of immune effector cells may be administered intravenously.

The immune effector cell or population of immune effector cells may be administered with one or more additional therapy, such as one or more additional therapeutic agents. The additional therapeutic agent may be an anti-tumour agent. The additional therapeutic may be an additional immune effector cell.

Combined administration of the immune effector cell or population with the additional therapeutic agent may be achieved in a number of different ways. All the components may be administered together in a single composition. Each component may be administered separately as part of a combined therapy.

For example, the immune effector cell or the population of immune effector cells of the invention may be administered before, after or concurrently with the additional therapeutic agent. The additional therapy may be chemotherapy, radiotherapy and/or surgery.

Prior to administration of the immune effector cell or population of immune effector cells of the invention, the subject may undergo lymphodepletion. Lymphodepletion may be achieved via administration to the subject with fludarabine, cyclophosphamide and/or bendamustine. Lymphodepletion may be carried out for at least about one day, such as about 2 days or about 3 days.

The biological activity and/or therapeutic efficacy of the administered immune effector cell or population of immune effector cells may be measured by known methods. For example, the method may comprise imaging, such as magnetic resonance imaging.

### Embodiments of the Invention

1. A method of optimising the effector function of an immune effector cell, wherein the immune effector cell comprises:
   a chimeric antigen receptor (CAR) capable of binding to an antigen on an antigen presenting cell (APC), wherein the CAR comprises a fusion protein comprising an extracellular antigen binding domain, a transmembrane domain, and an intracellular signalling domain, wherein the extracellular antigen binding domain and the transmembrane domain are linked by a hinge; and
   an accessory receptor capable of binding to a ligand on the APC, wherein the accessory receptor comprises an extracellular ligand binding domain and a transmembrane domain, wherein the extracellular ligand binding domain and the transmembrane domain are linked by a stalk;
   wherein the method comprises modifying the height of the extracellular antigen binding domain of the CAR and/or the height of the extracellular ligand binding domain of the accessory receptor from the membrane of the immune effector cell to optimise the effector function of the immune effector cell upon contact with the APC;
   optionally wherein the effector function is cell killing.
2. A method of identifying an improved immune effector cell, wherein the immune effector cell comprises:
   a chimeric antigen receptor (CAR) capable of binding to an antigen on an antigen presenting cell (APC), wherein the CAR comprises a fusion protein comprising an extracellular antigen binding domain, a transmembrane domain, and an intracellular signalling domain, wherein the extracellular antigen binding domain and the transmembrane domain are linked by a hinge; and
   an accessory receptor capable of binding to a ligand on the APC, wherein the accessory receptor comprises an extracellular ligand binding domain and a transmembrane domain, wherein the extracellular ligand binding domain and the transmembrane domain are linked by a stalk;
   wherein the method comprises:
   (a) modifying the height of the extracellular antigen binding domain of the CAR and/or the height of the extracellular ligand binding domain of the accessory receptor from the membrane of the immune effector cell; and
   (b) determining whether an immune effector cell expressing a modified CAR and/or a modified accessory receptor has improved effector function when compared to an immune effector cell expressing an unmodified CAR and/or unmodified accessory receptor;
   optionally wherein the effector function is cell killing.
3. The method of embodiment 1 or 2, comprising modifying the height of the extracellular antigen binding domain of the CAR and/or the height of the extracellular ligand binding domain of the accessory receptor such that the intermembrane distance spanned by the CAR-antigen complex is comparable with the intermembrane distance spanned by the accessory receptor-ligand complex.
4. The method of embodiment 3, wherein:
   (a) the intermembrane distance spanned by the CAR-antigen complex is within ≤5%, ≤10%, ≤15%, ≤20%, ≤25%, or ≤30%, or is the same as, the intermembrane distance spanned by the accessory receptor-ligand complex; and/or
   (b) the intermembrane distance spanned by the CAR-antigen complex is comparable with (e.g. within ≤5%, ≤10%, ≤15%, ≤20%, ≤25%, or ≤30%, or is the same as) the intermembrane distance spanned by the complex between a T cell receptor and its peptide-MHC antigen.
5. The method of embodiment 3 or 4, wherein the intermembrane distance spanned is about 14 nm.
6. The method of any one of the preceding embodiments, comprising:
   (a) reducing the height of the extracellular antigen binding domain of the CAR;
   (b) increasing the height of the extracellular antigen binding domain of the CAR;
   (c) reducing the height of the extracellular ligand binding domain of the accessory receptor;
   (d) increasing the height of the extracellular ligand binding domain of the accessory receptor;
   (e) reducing the height of the extracellular antigen binding domain of the CAR and increasing the height of the extracellular ligand binding domain of the accessory receptor; or
   (f) increasing the height of the extracellular antigen binding domain of the CAR and reducing the height of the extracellular ligand binding domain of the accessory receptor.
7 The method of any one of the preceding embodiments, wherein the height of the antigen binding domain of the CAR from the membrane of the immune effector cell is comparable with (e.g. within ≤5%, ≤10%, ≤15%, ≤20%, ≤25%, or ≤30%, or is the same as) the height of the extracellular antigen binding domain of a T cell receptor from the membrane of an immune effector cell (e.g. about 7 nm).
8. The method of any one of the preceding embodiments, wherein the height of the ligand binding domain of the accessory receptor from the membrane of the immune effector cell is about 7 nm.
9. The method of any one of the preceding embodiments, comprising introducing into the hinge of the CAR and/or the stalk of the accessory receptor, or replacing the hinge of the CAR and/or the stalk of the accessory receptor with, a sequence that physically increases the height of the ligand binding domain from the membrane of the immune effector cell.
10. The method of embodiment 9, wherein the sequence introduced into the hinge in the CAR comprises or consists of:
   (a) a fragment of the mucin-like extracellular sequence of CD43 as set out in SEQ ID NO: 62, e.g. between 4 and 234 amino acids, such as between 20 and 40 amino acids (e.g. as set out in any one of SEQ ID NOs: 53 to 61);
   (b) a folded polypeptide domain which is an immunoglobulin domain, such as an immunoglobulin constant domain, or a FNIII domain; and/or
   (c) a fragment of the CD28 hinge as set out in SEQ ID NO: 10 or the CD8a hinge as set out in SEQ ID NO: 43, optionally wherein the fragment is as set out in any one of SEQ ID NOs: 37 to 42, 46, 63 and 64.
11. The method of embodiment 9, wherein the sequence introduced into the stalk of the accessory receptor comprises or consists of a fragment of a mucin-like sequence, or a fragment or a derivative thereof, optionally wherein the mucin-like sequence is the mucin-like sequence of CD43 as set out in SEQ ID NO: 62, and optionally wherein the fragment is of between 4 and 234 amino acid residues in length, such as any one of SEQ ID NOs: 53 to 61.
12. The method of any one of the preceding embodiments, wherein the accessory receptor is an adhesion receptor, e.g. CD2.
13. The method of any one of the preceding embodiments, wherein the antigen is a peptide-MHC complex, CD19, mesothelin, BCMA, CD22, EGFR, or EGFRvIII, optionally wherein the peptide in the peptide-MHC complex is a fragment of NY-ESO 1.
14. An immune effector cell obtained or obtainable by the method of any one of the preceding embodiments.
15. A chimeric antigen receptor (CAR) capable of binding to an antigen on an antigen presenting cell (APC), comprising a fusion protein comprising an extracellular antigen binding domain, a transmembrane domain, and an intracellular signalling domain, wherein the extracellular antigen binding domain and the transmembrane domain are linked by a hinge, wherein the hinge comprises or consists a sequence that physically increases the height of the extracellular antigen binding domain from the membrane of the immune effector cell, wherein the sequence comprises a mucin-like sequence, one or more folded polypeptide domains or a fragment of the CD28 as set out in SEQ ID NO: 10 or the CD8a hinge as set out in SEQ ID NO: 43.
16. An accessory receptor capable of binding to a ligand on the APC, comprising an extracellular ligand binding domain and a transmembrane domain, wherein the extracellular ligand binding domain and the transmembrane domain are linked by a stalk, wherein the stalk comprises or consists of a sequence that physically increases the height of the ligand binding domain from the membrane of the immune effector cell, optionally wherein the stalk comprises a mucin-like sequence.
17. An immune effector cell comprising the CAR of embodiment 15 and/or the accessory receptor of embodiment 16.
18. An immune effector cell comprising:
   a chimeric antigen receptor (CAR) capable of binding to an antigen on an antigen presenting cell (APC), wherein the CAR comprises a fusion protein comprising an extracellular antigen binding domain, a transmembrane domain, and an intracellular signalling domain, wherein the extracellular antigen binding domain and the transmembrane domain are linked by a hinge; and
   an accessory receptor capable of binding to a ligand on the APC, wherein the accessory receptor comprises an extracellular ligand binding domain and a transmembrane domain, wherein the extracellular ligand binding domain and the transmembrane domain are linked by a stalk;
   wherein:
   (a) the CAR and the accessory receptor are each of a size such that the intermembrane distance spanned by the CAR-antigen complex is comparable with the intermembrane distance spanned by the accessory receptor-ligand complex;
   (b) the hinge of the CAR comprises or consists of a sequence that physically increases the height of the antigen binding domain from the membrane of the immune effector cell, wherein the sequence comprises a mucin-like sequence, one or more folded polypeptide domains or a fragment of the CD28 as set out in SEQ ID NO: 10 or the CD8a hinge as set out in SEQ ID NO: 43; and/or
   (c) the stalk of the accessory receptor comprises a sequence that physically increases the height of the extracellular ligand binding domain from the membrane of the immune effector cell.
19. The immune effector cell of embodiment 18, wherein:
   (a) the intermembrane distance spanned by the CAR-antigen complex is within ≤5%, ≤10%, ≤15%, ≤20%, ≤25%, or ≤30%, or is the same as the intermembrane distance spanned by the accessory receptor-ligand complex; and/or
   (b) the intermembrane distance spanned by the CAR-antigen complex or the accessory receptor-ligand complex is comparable with (e.g. within ≤5%, ≤10%, ≤15%, ≤20%, ≤25%, or ≤30%, or is the same as) the intermembrane distance spanned by the complex between a T cell receptor and its corresponding peptide-MHC antigen.
20. The immune effector cell of embodiment 18 or 19, wherein the hinge of the CAR comprises or consists of a sequence that physically increases the height of the antigen binding domain from the membrane of the immune effector cell, wherein the sequence comprises a mucin-like sequence, one or more folded polypeptide domains or a fragment of the CD28 as set out in SEQ ID NO: 10 or the CD8a hinge as set out in SEQ ID NO: 43.
21. The immune effector cell of embodiment 18 or 19, wherein the stalk of the accessory receptor comprises a sequence that physically increases the height of the extracellular ligand binding domain from the membrane of the immune effector cell.
22. The immune effector cell of embodiment 18 or 19, wherein:
   (a) the hinge of the CAR comprises or consists of a sequence that physically increases the height of the antigen binding domain from the membrane of the immune effector cell, wherein the sequence comprises a mucin-like sequence, one or more folded polypeptide domains or a fragment of the CD28 as set out in SEQ ID NO: 10 or the CD8a hinge as set out in SEQ ID NO: 43; and
   (b) the stalk of the accessory receptor comprises a sequence that physically increases the height of the extracellular ligand binding domain from the membrane of the immune effector cell.
23 The immune effector cell of any one of embodiments 17 to 22, wherein the immune effector cell is a T cell, an NK cell, an NKT cell, a phagocyte or a macrophage, optionally wherein the T cell is a CD4-CD8+ T cell.
24. A method for identifying an improved immune effector cell, comprising determining whether the immune effector cell of any one of embodiments 17 to 23 has improved effector function when compared to an immune effector cell expressing an unmodified CAR and/or unmodified accessory receptor.
25. The immune effector cell of any one of embodiments 17 to 23, the CAR of embodiment 15 or the accessory receptor of embodiment 16, for use in a method of treating cancer in a subject, optionally where the cancer is haematological malignancy or B cell cancer.

### Other

It is to be understood that different applications of the disclosed CARs, immune effector cells, or pharmaceutical compositions of the invention may be tailored to the specific needs in the art. It is also to be understood that the terminology used herein is for the purpose of describing particular embodiments of the invention only, and is not intended to be limiting.

In addition, as used in this specification and the appended claims, the singular forms “a”, “an”, and “the” include plural references unless the content clearly dictates otherwise. Thus, for example, reference to “a CAR” includes two or more “CARs”.

Furthermore, when referring to “≥x” herein, this means equal to or greater than x. When referred to “≤x” herein, this means less than or equal to x.

For the purpose of this invention, in order to determine the percent identity of two sequences (such as two polynucleotide or two polypeptide sequences), the sequences are aligned for optimal comparison purposes (e.g. gaps can be introduced in a first sequence for optimal alignment with a second sequence). The nucleotide or amino acid residues at each position are then compared. When a position in the first sequence is occupied by the same nucleotide or amino acid as the corresponding position in the second sequence, then the nucleotides or amino acids are identical at that position. The percent identity between the two sequences is a function of the number of identical positions shared by the sequences (i.e., % identity=number of identical positions/total number of positions in the reference sequence×100).

Typically, the sequence comparison is carried out over the length of the reference sequence. For example, if the user wished to determine whether a given (“test”) sequence is 95% identical to SEQ ID NO: 3, SEQ ID NO: 3 would be the reference sequence. To assess whether a sequence is at least 95% identical to SEQ ID NO: 3 (an example of a reference sequence), the skilled person would carry out an alignment over the length of SEQ ID NO: 3, and identify how many positions in the test sequence were identical to those of SEQ ID NO: 3. If at least 95% of the positions are identical, the test sequence is at least 95% identical to SEQ ID NO: 3. If the sequence is shorter than SEQ ID NO: 3, the gaps or missing positions should be considered to be non-identical positions.

The skilled person is aware of different computer programs that are available to determine the homology or identity between two sequences. For instance, a comparison of sequences and determination of percent identity between two sequences can be accomplished using a mathematical algorithm. In an embodiment, the percent identity between two amino acid or nucleic acid sequences is determined using the Needleman and Wunsch (1970) algorithm which has been incorporated into the GAP program in the Accelrys GCG software package (available at http://www.accelrys.com/products/gcg/), using either a Blosum 62 matrix or a PAM250 matrix, and a gap weight of 16, 14, 12, 10, 8, 6, or 4 and a length weight of 1, 2, 3, 4, 5, or 6.

By “specific” or “specifically binds”, it is meant that the antigen-binding region of a CAR binds to one or more antigenic determinants of the desired target antigen and does not bind to with other polypeptides. For example, a CAR specific for CD19 binds to with an antigen of CD19 but does not bind to an antigen of a different polypeptide such as bovine serum albumin. A CAR may specifically bind if it binds to the target antigen with at least a 10-fold stronger affinity when compared to binding an antigen of a different polypeptide such as bovine serum albumin, preferably, at least with at least a 100-fold stronger affinity. Methods for measuring the affinity of binding are well known in the art.

All publications, patents and patent applications cited herein, whether supra or infra, are hereby incorporated by reference in their entirety.

The following examples illustrate the invention.

### Example 1—Identify the Mechanism Underlying the Failure of CARs to Exploit CD2 Adhesion

### Determining the Contributions of Antigen Receptor Size and Signalling to Antigen Sensitivity

The inventors generated a modified CAR, referred to herein as Fab CAR. The Fab-CAR is a fusion protein with antibody derived variable domains coupled to TCR constant domains, followed with the CD28 transmembrane domain and then the TCR-ζ chain cytoplasmic region (FIG. 2). Importantly, this construct omitted the additional extracellular hinge that is often incorporated into CARs, which makes them larger than the TCR.

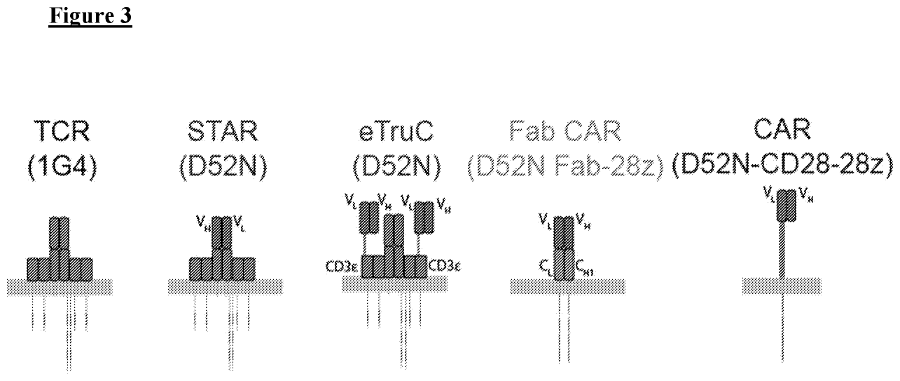

The activity of the following receptors were tested: 1G4 TCR, STAR (D52N), eTruC (D52N), Fab-CAR(D52N) and CAR (D52N-CD28-28z) (FIG. 3). The D52N single chain variable fragment (scFv) binds the same peptide-MHC complex [the C9V variant (9V) of the NY-ESO-1157-165 peptide presented on HLA-A*02:01] that is recognised by the 1G4 TCR (4).

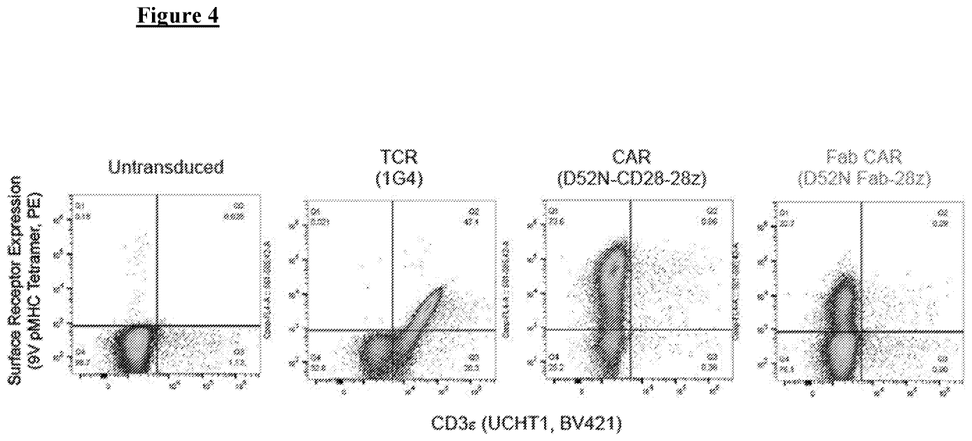

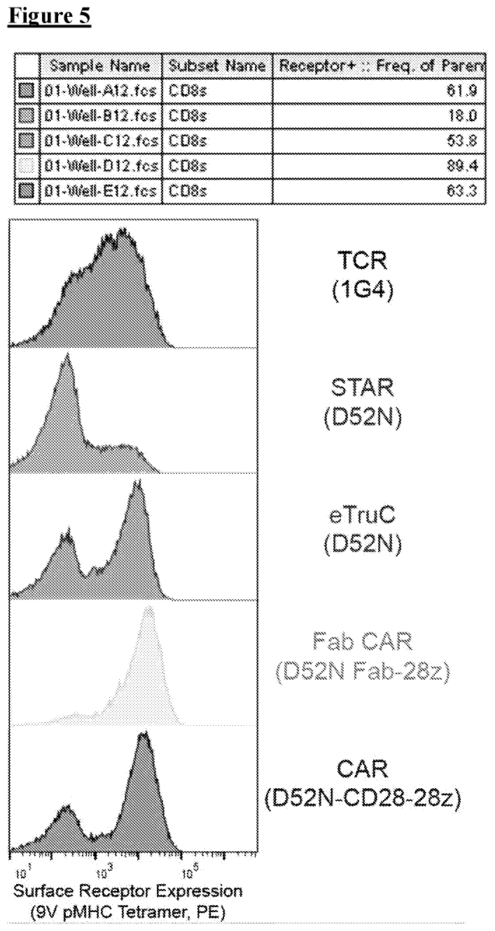

The receptors were lentivirally transduced in to Jurkat cells or primary human CD8+ T cells, and were successfully expressed (see FIGS. 4, 5 and 6). Interestingly, FIG. 4 shows the standard CAR (D52N-CD28-28z), and the Fab-CAR (D52N-Fab-28z) are expressed (y-axis) without upregulating CD3 (x-axis). Hence, these receptors can be expressed independent of the TCR-CD3 complex, which is not the case for the 1G4 TCR.

The T2 target cell was loaded with the indicated concentration of peptide antigen before being incubated with T cells expressing different antigen receptors and/or different sizes of CD2. The dose-responses and EC50 values (antigen sensitivity; the concentration of antigen required to elicit 50% of the maximum response) are shown in FIGS. 6A to 6C. In FIG. 7, cytokine production is shown when using primary T cells.

It can be seen that STARs, which have the same size and signalling machinery as the TCR, have comparably high antigen sensitivity. In contrast TruCs, which have the same signalling machinery as the TCR but differ in size, exhibited lower antigen sensitivity, indicating that comparable size is necessary. Consistent with this, and strikingly, the Fab-CAR, which was expressed independent of the TCR-CD3 complex and shared a similar size with the TCR and STAR, achieved similar antigen sensitivities and cytokine productivity to TCR and STAR but achieved higher antigen sensitivity and cytokine production compared to the CAR and eTRuC.

The data therefore show that comparable size rather than signalling machinery is necessary to enable CARs to achieve the same high antigen sensitivity as the TCR. The data are supported by previous findings that T cell antigen recognition is dependent on the dimensions of an accessory receptor-ligand complex (11), that the dimensions of the CD2/ligand complex matches the dimensions of the TCR/pMHC complex (14 nm) (3,6), and that varying the dimensions of either the CD2/ligand or the TCR/pMHC complexes (7).

### Materials and Methods

### Plasmid Constructs

Generation of lentivirus infectious particles was conducted using a third generation system, which included: the (i) pMD2.G (Addgene #12259), (ii) pRSV-Rev (Addgene #12253), and (iii) pMDLg/pRRE (Addgene #12251) packaging plasmid. Diverse lentiviral transfer vectors, encoding receptors that bind the NY-ESO-1/HLA-A*02 pMHC complex were used. The coding sequences were contained either in a pLEX_307 (Addgene #41392) or a pLEX307-NeoR (Addgene #134365) backbone.

### Receptors

The 1G4 T cell receptor (TCR), has a variable alpha (SEQ ID NO. 1) and constant alpha (SEQ ID NO. 2) domain, a P2A self-cleaving peptide (SEQ ID NO. 3), followed by a variable beta (SEQ ID NO. 4) and constant beta (SEQ ID NO. 5) domain. The variable domains confer specificity.

The D52N-CD28.H-28z CAR, also referred to herein as D52N-CD28-28z, has: a leader sequence (SEQ ID NO. 6), the variable heavy domain of the D52N scFv (SEQ ID NO. 7), a linker (SEQ ID NO. 8), the variable light domain of the D52N scFv (SEQ ID NO. 9), a CD28-derived hinge (SEQ ID NO. 10), a CD28 transmembrane domain (SEQ ID NO. 11), a CD28 cytoplasmic domain (SEQ ID NO. 12), and a zeta chain (CD247) signalling tail (SEQ ID NO. 13).

A CAR similar to D52N-CD28.H-28z CAR, except for a CD8a hinge instead of a CD28 hinge may be used. Such a CAR may have a leader sequence (SEQ ID NO. 6), variable heavy domain of the D52N scFv (SEQ ID NO. 7), a linker (SEQ ID NO. 8), the variable light domain of the D52N scFv (SEQ ID NO. 9), a CD8a-derived hinge (SEQ ID NO. 43), a CD8a transmembrane domain (SEQ ID NO. 44), and a zeta chain (CD247) signalling tail (SEQ ID NO. 13). An alternative leader sequence may be used, e.g. a GM-CSF leader sequence as set out in SEQ ID NO: 65.

The D52N-ε-TRuC, also referred to herein as eTruC (D52N), has: a leader sequence (SEQ ID NO. 14), the D52N scFv (both heavy and light domains) (SEQ ID NO. 15), a glycine-serine linker (SEQ ID NO. 16), the extracellular, helical, and cytoplasmic regions of human CD3E (SEQ ID NO. 17). As is well known in the art, a TRUC is a T cell receptor fusion construct, e.g. see reference 12).

The D52N-STAR, also referred to herein as STAR (D52N), has: a leader sequence (SEQ ID NO. 14), the variable heavy domain of the D52N scFv (SEQ ID NO. 7), a murine constant alpha TCR domain (SEQ ID NO. 18), a P2A self-cleaving peptide (SEQ ID NO. 3), a leader sequence (SEQ ID NO. 19), the variable light domain of the D52N scFv (SEQ ID NO. 9), and a murine constant beta TCR domain (SEQ ID NO. 20). As is well known in the art, a STAR is a synthetic T cell receptor and antigen receptor, e.g. see reference 13)

The D52N-Fab-CAR has: a leader sequence (SEQ ID NO. 14), the variable heavy domain of the D52N scFv (SEQ ID NO. 7), an IgG1-CH1 domain (SEQ ID NO. 21), a linker (SEQ ID NO. 22), a CD28 transmembrane domain (SEQ ID NO. 11), a CD28 cytoplasmic domain (SEQ ID NO. 12), a zeta chain (CD247) signalling tail (SEQ ID NO. 13), a GSG stuffer (SEQ ID NO. 23), a P2A self-cleaving peptide (SEQ ID NO. 3), a leader sequence (SEQ ID NO. 14), the variable light domain of the D52N scFv (SEQ ID NO. 9), an IgG1-CL domain (SEQ ID NO. 24), a linker (SEQ ID NO. 22), a CD28 transmembrane domain (SEQ ID NO. 11), a CD28 cytoplasmic domain (SEQ ID NO. 12), and a zeta chain (CD247) signalling tail (SEQ ID NO. 13).

### Production of Lentiviral Supernatants

293T cells (ATCC CRL-3216) were plated at a 50% confluency in a volume of 3 mL using a 6-well plate. Cells were allowed to attach overnight and, the following morning, transfected with a mixture of packaging plasmids (950 ng of pRSV-Rev, 370 ng of pMD2.G, 950 ng of pMDLg/pRRE, and 1000 ng of the corresponding lentiviral transfer plasmid). Transfection was executed using the X-tremeGENE HP Transfection Reagent using a ratio of 3 μL of transfection reagent to 1 μg of DNA. 293T cells were then incubated at 37 °C 10% CO₂ (v/v) for 48 h, and the lentiviral supernatant filtered through a 0.45 μm cellulose acetate syringe filter. The supernatant was used to transduce either Jurkat or primary CD8+ T cells. Lentiviral supernatants were applied either directly or previous concentration using the Lenti-X concentrator reagent (Takara Bio) according to manufacturer's instructions. Stored supernatant was maintained at −80 °C.

### Cells

The follow cell lines were used: 293T cells (ATCC CRL-3216), T2 cells (ATCC CRL-1993), Jurkat E6.1 NFkB/eGFP cells (which were derived by lentiviral transduction of the pSIRV—NF-κB plasmid (Addgene #118093) into the Jurkat E6-1 clone (ATCC TIB-152), Jurkat E6.1 TCRα−β− cells.

Human leukocyte cones were acquired, and CD8+ T cells were isolated. Isolated CD8+T lymphocytes mixed with human anti-CD3/CD28 Dynabeads at a 1:1 ratio of cells to beads. Cells were rested overnight and, the following morning, 1.5 million CD8+ T cells transduced with the corresponding lentiviral supernatant encoding the receptor of interest. Seventy-two hours post-transduction, cells were subjected either to puromycin (pLEX_307 backbone) or G418 (pLEX307-NeoR backbone) selection.

### T Cell Stimulation Assays

The T2 suspension cell line was used as a surrogate APC, onto which the 9V NY-ESO-1 peptide was loaded. Chiefly, 3.3×10⁴ T2 cells were seeded in a V-bottom 96-well plate in a volume of 110 μL of full RPMI. Serial dilutions of the 9V peptide were prepared also using full RPMI. 110 μL of the diluted 9V peptide were then added to the T2 cell suspension, and the cells incubated for 90 min at 37 °C and 5% CO2 (v/v). The T2 cells were then washed with full RPMI and resuspended in 110 μL of this media. A total of 30,000 T2 cells (corresponding to 100 μL) were transferred to a U-bottom 96-well plate. After this, a total of 60,000 CD8+ T cells (in a volume of 100 μL) were added to each well, establishing a simple 2:1 effector:target ratio. Plates were gently centrifuged (15 g for min) to promote contact between the T cells and the APCs. Co-cultures were conducted during 20 h, with the cells being incubated at 37 °C and 5% CO2 (v/v).

### Flow Cytometry

After an experiment was concluded, cells were transferred to a 96-well plate with a V bottom and centrifuged at 520 g for 5 min at 4 °C. Cells were first stained with a fixable viability dye (Zombie near-infrared, 1:500 working dilution) in a volume of 50 μL of PBS. Samples were then stained with conjugated flow cytometry antibodies, which were previously diluted in 50 μL of PBS. Working dilutions ranged from 1:200 for commercial antibodies to 1:1000 for pMHC 9V tetramers. Cells were incubated for 30 min, washed, and resuspended in PBS with 1% BSA. Sample acquisition was conducted in a BD X-20 or Cytoflex cytometer, with analyses conducted with the FlowJo suite.

Of note, detection of the multiple receptors was conducted with fluorescent pMHC tetramers. The tetramers were composed of biotinylated, refolded 9V pMHC molecules complexed with PE streptavidin. Fluorescent tetramers were prepared by vigorously mixing 66.6 μg of monomeric 9V pMHC with step-wise additions of 10 μL of PE streptavidin every 10 min (10 additions of PE streptavidin over 100 min).

### Cytokine Measurements

Following the co-culture experiments, supernatants were assayed for levels of the IFN-γ and IL-2 using a commercial kit provided by Thermo Fisher Scientific.

### Example 2—Preparation of Modified Adhesion Receptors to Enhance the Antigen Sensitivity of CARS

This Example investigates engineering modified adhesion receptor-ligand complexes with varying dimensions to maximise their ability to enhance CAR-antigen complexes.

### Construct Elongation Variants of CD2 and Screen for Ability to Impact Sensitivity.

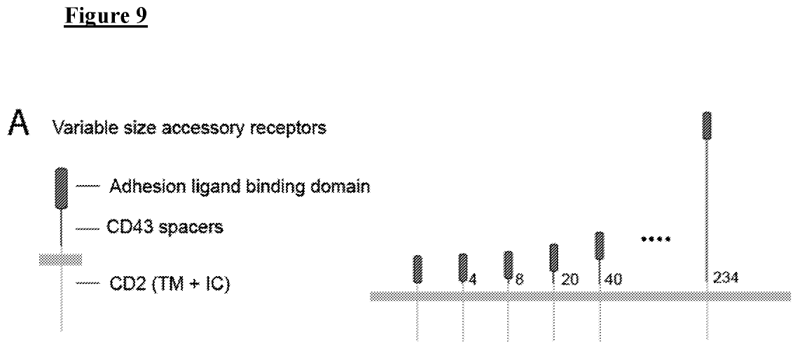

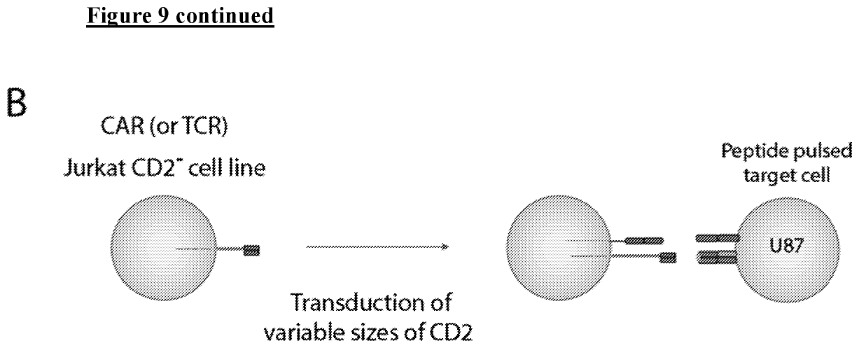

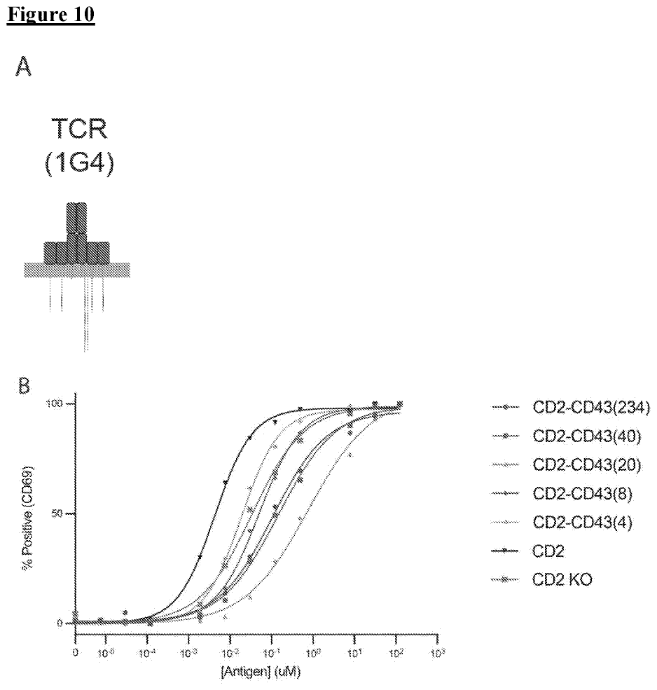

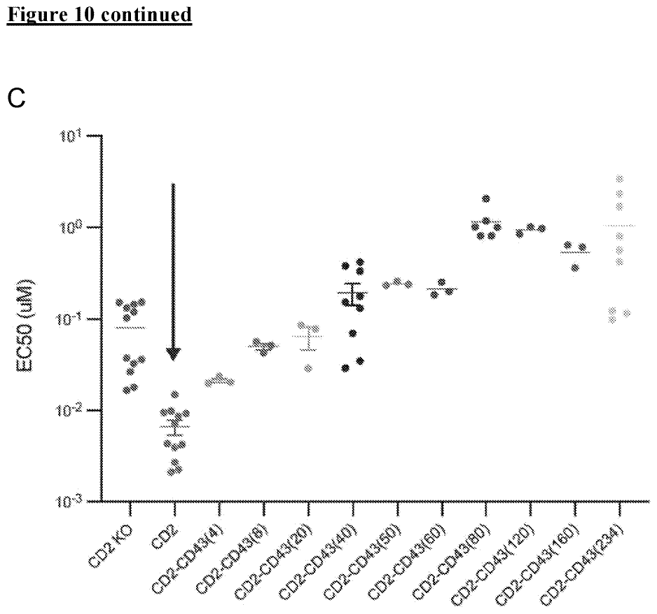

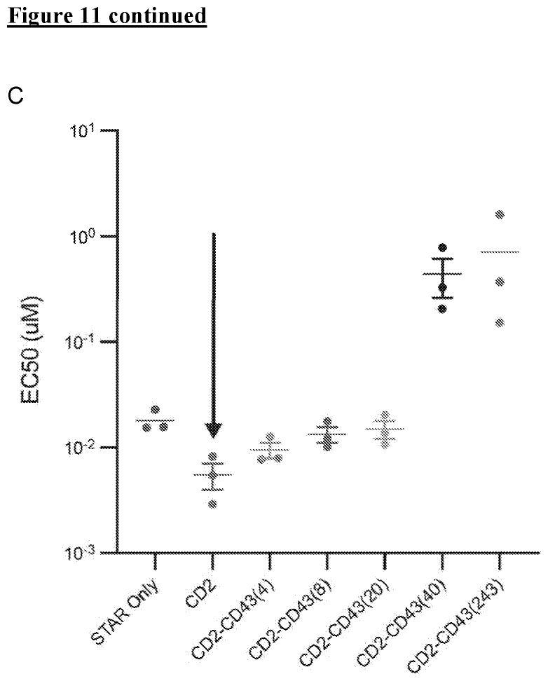

A panel of elongated CD2 constructs were prepared by inserting as spacers different fragments of extracellular domain of the human mucin-like protein CD43 (FIG. 9A). Either the 1G4 TCR or standard CARs were expressed in a CD2⁻ E6.1 Jurkat T cell line before transducing different CD2 constructs (FIG. 9B).

Whereas, as expected, wild-type CD2 greatly increased the antigen sensitivity of the TCR, elongation of CD2 with 4, 8, 20, 40, 50, 60, 80, 120, 160, or 234 amino acids from CD43 led to a progressive loss in antigen sensitivity (FIG. 10). Similarly, antigen sensitivity of the STAR was greatly increased with wild-type CD2, and elongation of CD2 with 4, 8, 20, 40 or 234 amino acids led to a progressive loss in antigen sensitivity (FIG. 11).

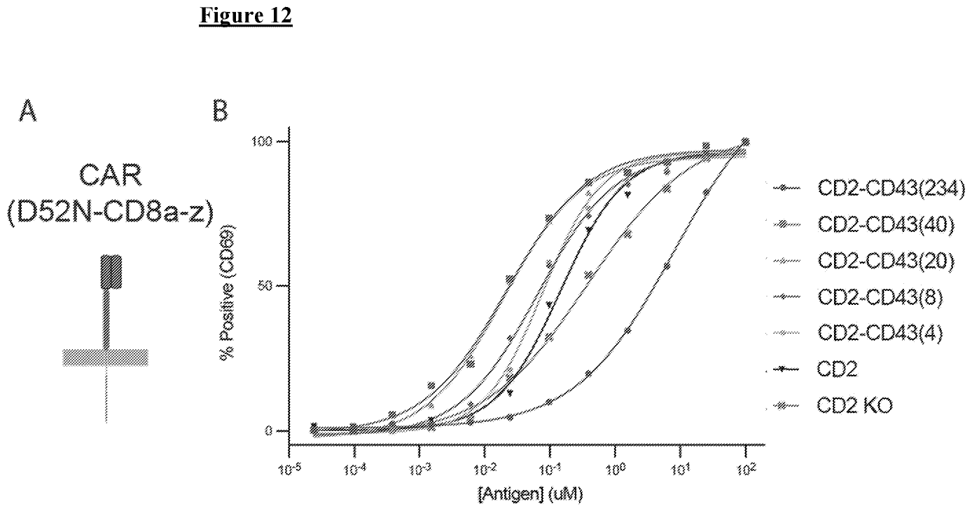

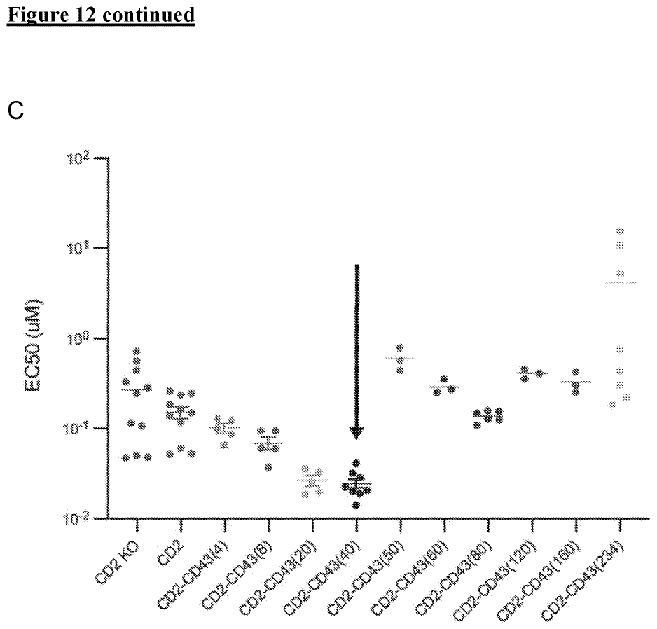

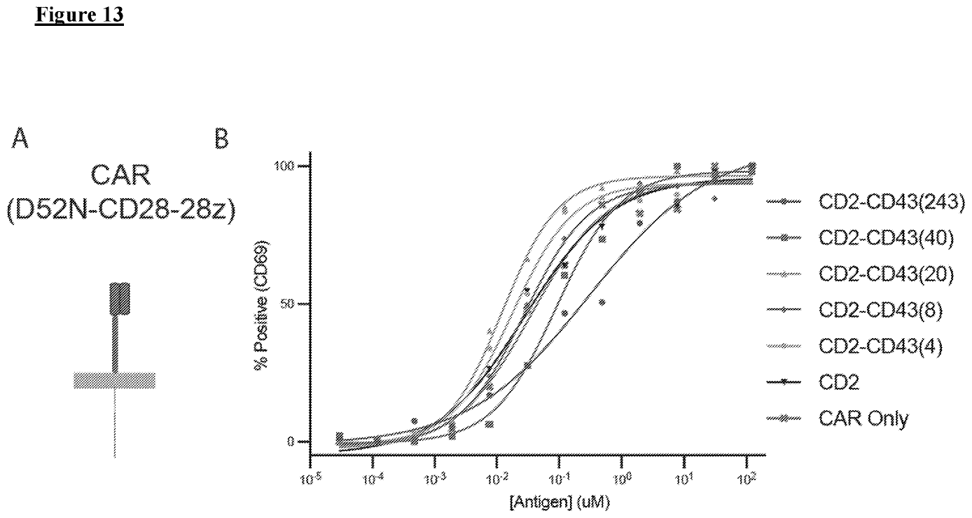

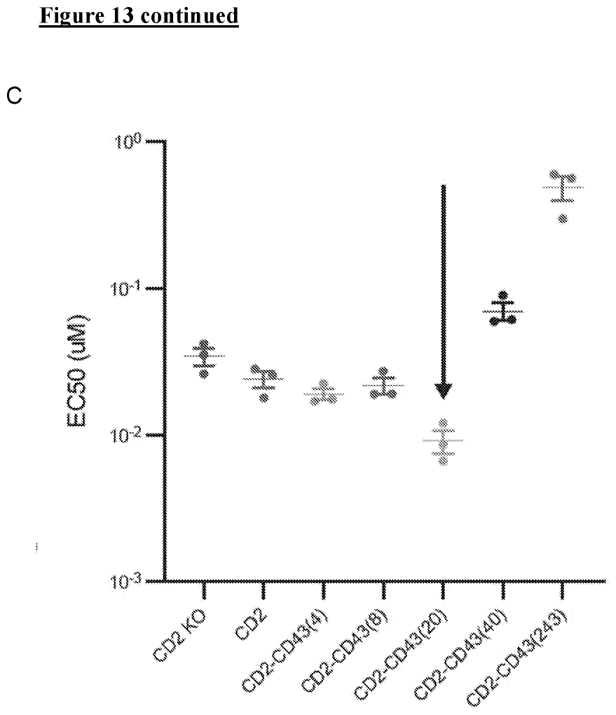

In striking contrast, wild-type CD2 had only a modest impact on CAR antigen sensitivity, whereas elongating CD2 increased antigen sensitivity, with the biggest increase achieved with an intermediate length CD2 construct (CD2-CD43(40) or CD2-CD43(20)) (FIGS. 12 and 13).

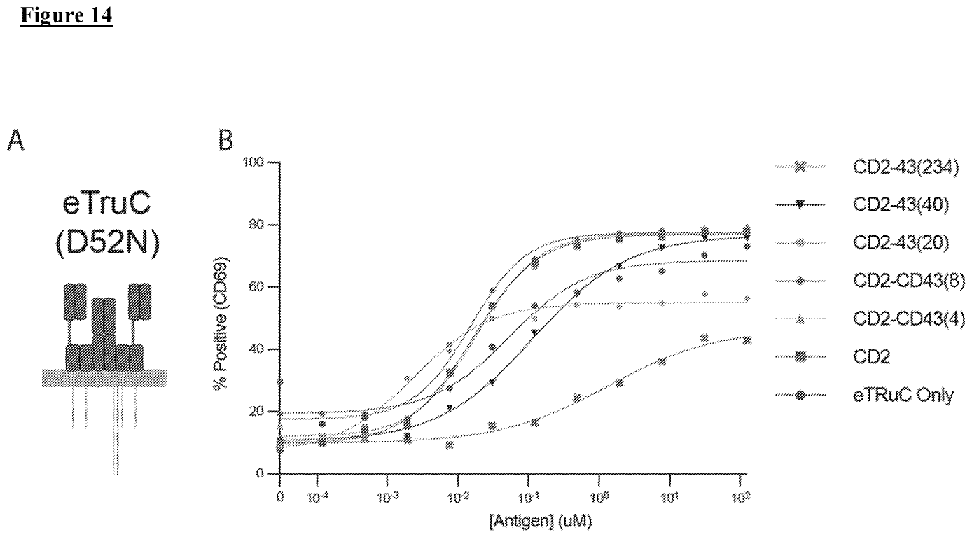

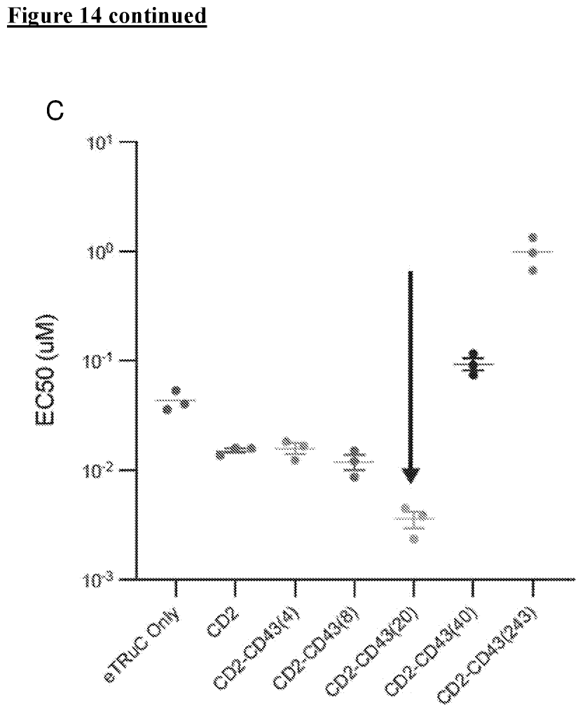

Wild-type CD2 increased the antigen sensitivity of eTruC. Elongation of CD2 increased the antigen sensitivity, with the biggest increase achieved with an intermediate length CD2 construct (CD2-CD43 (20)) (FIG. 14). Based on the size of the eTruC relative to the TCR, and the results of elongating CD2 in combination with CARs, this result could be expected based on the inventor's findings.

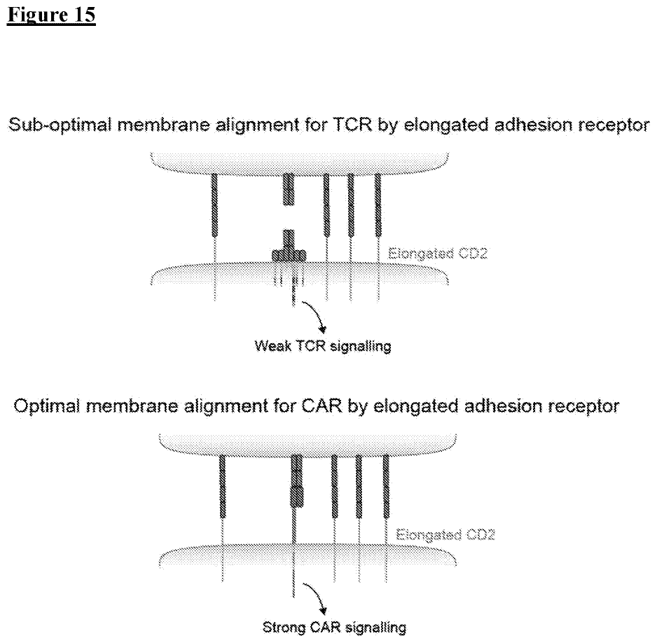

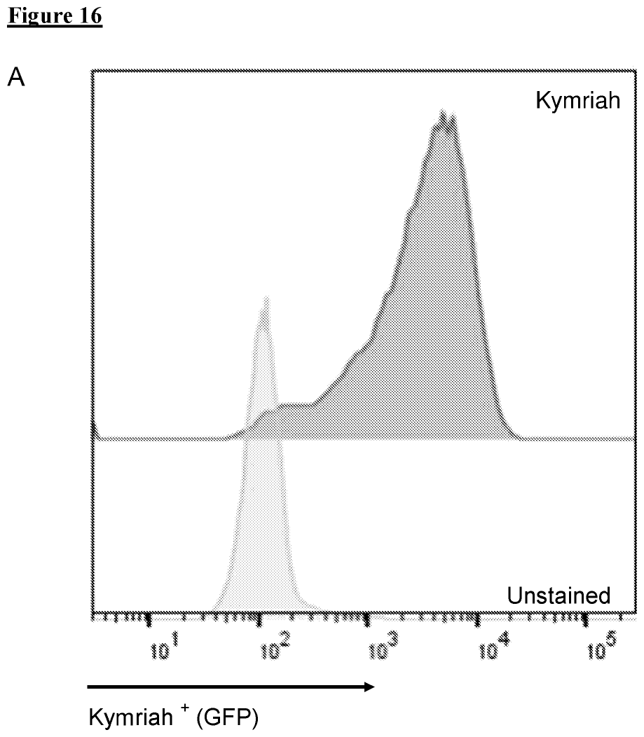

Therefore, the data show that the sensitivity of CARs can be increased by increasing the size of the accessory-ligand complex (e.g. CD2/CD58 complex). It also confirms that it is the greater size of the CAR-antigen complex that limits its ability to exploit CD2-CD58 (FIG. 15). The CD2-CD43 (234) antagonises TCR and CAR antigen recognition, which is attributed to membranes being held too far apart for antigen binding.

### Materials & Methods

### Production of Elongated CD2

Elongated CD2 variants were produced by fusing the extracellular portion of CD2 (SEQ ID NO: 49) with that of CD43 using a short linker sequence (GGGS; SEQ ID NO: 50), followed by the CD2 transmembrane domain (SEQ ID NO: 51) and the CD2 intracellular domain (SEQ ID NO: 52). From the full-length sequence consisting of the entire CD43 extracellular portion shorter variants were produced using site directed mutagenesis leaving a number of amino acids from the C terminal end of the CD43 domain (e.g. SEQ ID NOs 53 to 62). For example, CD2-CD43(20) contains the CD2 extracellular portion fused to the 20 amino acids proximal to the C terminal end of the CD43 extracellular portion. The elongated CD2 variants are provided in SEQ ID NOs: 27 to 36, each of which also includes the CD2 signal peptide (SEQ ID NO: 47) and HA tag (SEQ ID NO: 48).

### Cell Lines

Jurkat TCRα−β− cells were a gift from Simon J. Davis (Oxford) and were cultured in RPMI 1640 10% FBS (v/v) with penicillin-streptomycin (100 U/mL and 100 mL, respectively) at 37 °C and 5% CO2.

### The Endogenous Surface Protein CD2 was Knocked Out Using CRISPR/Cas9.

Co-Culture with U87 Cells

25000 U87 cells were seeded in a tissue culture treated flat-bottom 96 well plate and grown overnight. On the following day the media was removed from these cells and they were incubated with peptides prepared to the appropriate concentration in complete DMEM (DMEM supplemented with 10% v/v FBS, 100 Units/ml penicillin, 100 μg/ml streptomycin) for 1 hour at 37 °C.

Peptide containing media was then removed and 50,000 T cells per well were added. The co-culture was then spun for 2 minutes at 50×g, and incubated for 4 hours at 37 °C. After this period a fraction of supernatant was removed for cytokine ELISAs and stored at −20 °C. EDTA was added to the remaining supernatant (final concentration 2.5 μM) and cells were detached by pipetting.

Cells were stained in PBS 1% BSA for CD45 (Clone HI30, dilution 1:200), CD69 (Clone FN50, dilution 1:200) and 4-1BB (Clone 4B4-1, dilution 1:200) as well as with PE-conjugated tetrameric pMHC (dilution 1:500). Stained cells were either analysed immediately or fixed with 1% formaldehyde in PBS and analysed on the following day. T cells were discriminated from U87 cells by CD45 staining and/or an assessment of size and complexity. Single T cells were identified on the basis of size and subsequent analysis performed on this population.

### Example 3—Impact of Variable Sizes of CD2 on Antigen Sensitivity of Tisagenlecleucel (Kymriah)

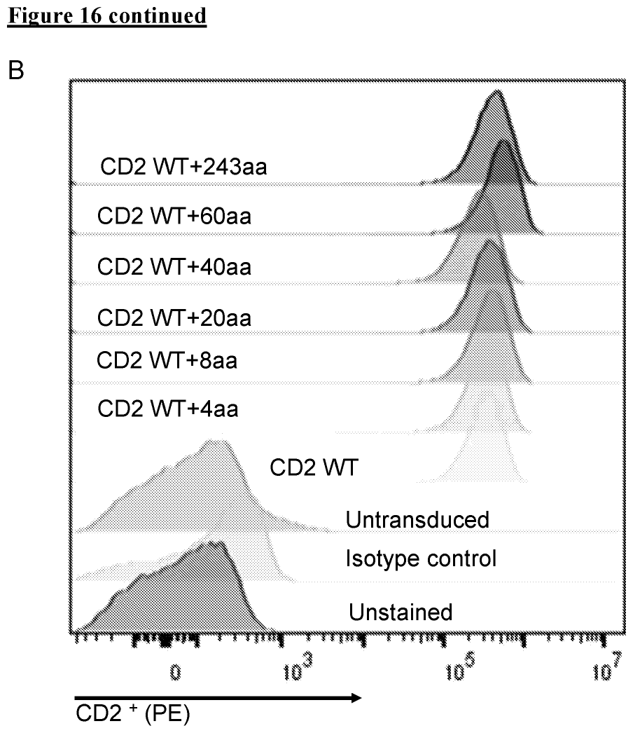

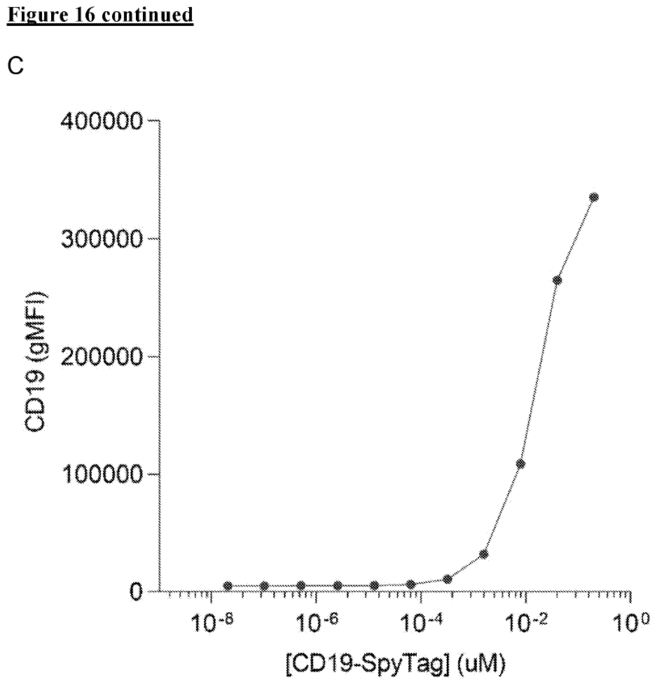

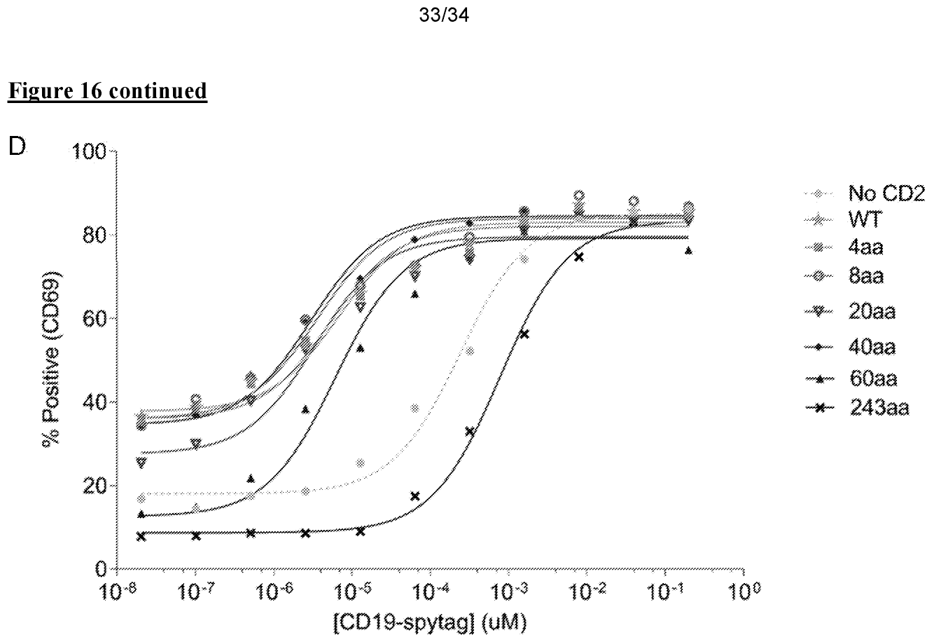

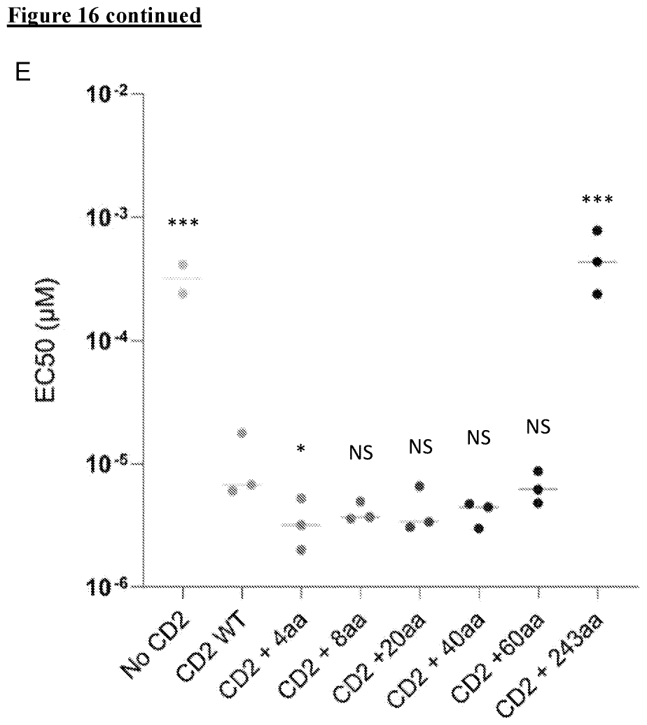

This Example examines the impact of variable sizes of CD2 on Kymriah, a clinically-approved CAR targeting the surface antigen CD19. Kymriah was expressed in CD2⁻ Jurkat T cells (FIG. 16A) together with expression of variable sizes of CD2. Cells were sorted for matched expression levels (FIG. 16B).

To examine the antigen sensitivity of Kymriah, the surface levels of CD19 on B cells was manipulated as described in reference 14 using Nalm6 CombiCells. These cells are CD19⁻ and express the protein Spycatcher, which forms a spontaneous covalent bond with Spytag, on their cell surface. By adding different concentrations of purified Spytag-CD19 in solution, Nalm6 CombiCells were produced with different levels of surface CD19 (FIG. 16C).

Each Jurkat line was co-cultured with Nalm6 CombiCells loaded with different levels of CD19 (FIGS. 16D, 16E). Compared to T cells that lacked CD2, it was found that wild-type CD2 produced a large improvement in antigen sensitivity (decrease in EC50) but increasing the size of CD2 by 4 amino acids led to a further improvement of antigen sensitivity. Further increasing the size of CD2 led to progressive decreases in antigen sensitivity with the longest variant displaying sensitivity that was similar to Jurkat T cells that lacked CD2.

Taken together, these results highlight that even modest elongations of CD2 can improve the antigen sensitivity of a CAR (Kymriah) targeting a folded antigen (CD19).

### Production of Kymriah Expressing Jurkat T Cells

HEK 293T cells were seeded in DMEM supplemented with 10% FBS and 1% penicillin/streptomycin in 6-well plates to reach 60-80% confluency on the following day. Cells were transfected with 0.25 pRSVRev (Addgene, 12253), 0.53 μg pMDLg/pRRE (Addgene, 12251), 0.35 μg pMD2.G (Addgene, 12259), and 0.8 μg of transfer plasmid expressing the Kymriah CAR using 5.8 X-tremeGENE HP (Roche). Media was replaced after 16 hours and supernatant harvested after a further 24 hours by filtering through a 0.45 μm cellulose acetate filter. Supernatant from one well of a 6-well plate was used to transduce 1 million CD2⁻ Jurkat T cells. Jurkat T cells were sorted for expression of the Kymriah CAR, then further cultured at a density of 0.3 million/ml.

Virus was prepared using HEK cells as described above. Kymriah-expressing CD2⁻ Jurkat T cells were transduced with WT CD2 or one of 6 variable size CD2 molecules. Jurkat T cells were sorted for matching level of expression of CD2 proteins then further cultured at a density of 0.3 million/ml.

### Coupling of Ligands to Nalm6 Cells

30,000 Nalm6 CombiCells were seeded in a TC-coated 96-well round bottom plate and incubated overnight at 37 C, 5% CO2. On experiment day, cells were transferred into a TC-coated 96-well V-bottom plate and spun down for 5 min at 520 g. Spytag-CD19 ligands were diluted to required concentration in complete RPMI (10% FCS, 1% Penicillin-Streptomycin). Existing media was removed from the cells and the diluted ligands added in a volume of 50 μl, and incubated for 40 minutes at 37 C, 5% CO2. The cells were then washed twice with complete RPMI.

### Co-Culture Assays Kymriah Jurkat T Cells and Nalm6 CombiCells

Kymriah Jurkat T cells were counted, and washed once in complete RPMI. 50,000 Jurkat T cells in 200 μl complete RPMI were added to Nalm6 CombiCells coupled with Spytag-CD19 and transferred into a 96-well round-bottomed plate. The cells were then incubated at 37 C, 5% CO₂ for 6 hours.

### Flow Cytometry—Detection of Spytag-CD19

Straight after ligand coupling and subsequent washing, the Nalm6 CombiCells were transferred to a v-bottom plate and spun for 5 minutes at 500 g, 4 C. The cells were washed once with PBS-BSA 1% for 5 minutes at 500 g, 4 C. To detect ligands, fluorescently conjugated antibodies against proteins of interest were diluted in PBS-BSA (1%), at a 1:200 dilution and added at a volume of 50 μl to the cells. The cells were re-suspended and incubated for 20 minutes at 4 C in the dark. The cells were washed twice in PBS, and re-suspended in 60 μl PBS, before running on a flow cytometer.

### Flow Cytometry—Detection of Kymriah Jurkat T Cell Activation

At the end of the stimulation assay, Kymriah Jurkat cells were then transferred to a v-bottom plate and washed once in 200 μl PBS 1% BSA (500 g, 4 C, 5 minutes). Antibodies against the activation marker CD69 were diluted in PBS 1% BSA at a 1:200 dilution. 50 μl of this staining solution was to the cells, before incubating them for 20 minutes at 4 C in the dark. The cells were washed twice in PBS, and re-suspended in 70 μl PBS, before running on a flow cytometer. The Nalm6 CombiCells could be distinguished from the Kymriah Jurkat T cells using their intrinsic GFP marker. Flow cytometry data was analysed using FlowJo (BD Biosciences).

### REFERENCES

1 Burton et al. (2021) Inefficient exploitation of accessory receptors reduces the sensitivity of chimeric antigen receptors. bioRxiv (preprint).
2 van der Merwe et al. (1995) Current Biology 5:74-84.
3 Demetriou, et al. (2020) Nature Immunology 21:1232-1243.
4 Maus et al., (2017) Mol Ther Oncolytics 11; 3:1-9.
5 Stewart-Jones et al., (2009) 106 (14) 5784-5788.
6 Kershaw et al. (2013) Nature Reviews Cancer, 13(8):525-41
7 Choudhuri et al., (2005) Nature, 436(7050):578-82.
8 Cyster et al., (1991) EMBO J. 10(4): 893-902.
9 Milstein et al., (2008) J Biol Chem 283(49): 34414-34422.
10 Rurik et al., (2022) Science 375(6576):91-96.
11 Wild et al. (1999) J Exp Med, 190(1):31-41.
12 Baeuerle et al. (2019) Nature communications, 10:2087.
13 Liu et al. (2021) Science Translational Medicine, 13(586): eabb5191.
14 Patel et al., (2023) bioRxiv, 2023-06 15.545075 (https://doi.org/10.1101/2023.06.15.545075)

Sequence listing

| SEQ ID | |
|---|---|
| 1 | 1G4 TCR variable alpha domain |
| 2 | 1G4 TCR constant alpha domain |
| 3 | Self cleaving P2A peptide |
| 4 | 1G4 TCR variable beta domain |
| 5 | 1G4 TCR constant beta domain |
| 6 | Leader sequence derived from IL-15 |
| 7 | N-term of D52N scFv (Vh region) |
| 8 | Linker of D52N scFv |
| 9 | C-term of D52N scFv (Vl region) |
| 10 | CD28 hinge (for use in conventional CARs) |
| 11 | CD28 transmembrane domain |
| 12 | CD28 cytoplasmic signalling tail |
| 13 | CD247 (zeta signalling-tail) |
| 14 | Leader sequence derived from GM-CSF |
| 15 | D52N scFv (Vh, linker, Vl) |
| 16 | Glycine-serine linker |
| 17 | Extracellular, helical, and cytoplasmic regions of human CD3E |
| 18 | Murine constant alpha TCR domain |
| 19 | Leader sequence derived from GM-CSF |
| 20 | Murine constant beta TCR domain |
| 21 | IgG1-CH1 domain |
| 22 | Gly4Ser linker |
| 23 | GSG linker |
| (GSG) | |
| 24 | IgG1-CL domain |
| 26 | CD2 |
| 27 | CD2-CD43(4) |
| 28 | CD2-CD43(8) |
| 29 | CD2-CD43(20) |
| 30 | CD2-CD43(40) |
| 31 | CD2-CD43(50) |
| 32 | CD2-CD43(60) |
| 33 | CD2-CD43(80) |
| 34 | CD2-CD43(120) |
| 35 | CD2-CD43(160) |
| 36 | CD2-CD43(234) |
| 37 | CD28 mini hinge (see FIG. 8) |
| 38 | CD28mini-CD43(4) |
| 39 | CD28mini-CD43(8) |
| 40 | CD28mini-CD43(12) |
| 41 | CD28 micro hinge |
| 42 | CD28 nano hinge |
| 43 | CD8a hinge |
| 44 | CD8a transmembrane domain |
| 46 | CD8a mini hinge |
| 47 | CD2 Signal Peptide |
| 48 | HA-Tag |
| 49 | CD2 Extracellular Domain |
| 50 | Linker |
| 51 | CD2 TM |
| 52 | CD2 Intracellular Domain |
| 53 | CD43(4) |
| 54 | CD43(8) |
| 55 | CD43(20) |
| 56 | CD43(40) |
| 57 | CD43(50) |
| 58 | CD43(60) |
| 59 | CD43(80) |
| 60 | CD43(120) |
| 61 | CD43(160) |
| 62 | CD43(234) |
| 63 | CD8a micro hinge |
| 64 | CD8a nano hinge |
| 65 | GM-CSF leader sequence |
| 66 | Peptide antigen recognised by D52N |

1. An immune effector cell comprising:
a chimeric antigen receptor (CAR) capable of binding to an antigen on an antigen presenting cell (APC), wherein the CAR comprises a fusion protein comprising an extracellular antigen binding domain, a transmembrane domain, and an intracellular signalling domain, wherein the extracellular antigen binding domain and the transmembrane domain are linked by a hinge; and
an accessory receptor capable of binding to a ligand on the APC, wherein the accessory receptor comprises an extracellular ligand binding domain and a transmembrane domain, wherein the extracellular ligand binding domain and the transmembrane domain are linked by a stalk;
wherein:
(a) the stalk of the accessory receptor comprises a sequence that physically increases the height of the extracellular ligand binding domain from the membrane of the immune effector cell;
(b) the hinge of the CAR comprises or consists of a sequence that physically increases the height of the antigen binding domain from the membrane of the immune effector cell, wherein the sequence comprises a mucin-like sequence, one or more folded polypeptide domains or a fragment of the CD28 as set out in SEQ ID NO: 10 or the CD8a hinge as set out in SEQ ID NO: 43; and/or
(c) the CAR and the accessory receptor are each of a size such that the intermembrane distance spanned by the CAR-antigen complex is comparable with the intermembrane distance spanned by the accessory receptor-ligand complex.

2. The immune effector cell of claim 1, wherein the intermembrane distance spanned by the CAR-antigen complex is within ≤5%, ≤10%, ≤15%, ≤20%, ≤25%, or ≤30%, or is the same as the intermembrane distance spanned by the accessory receptor-ligand complex.

3. The immune effector cell of claim 1 or 2, wherein the intermembrane distance spanned by the CAR-antigen complex or the accessory receptor-ligand complex is comparable with (e.g. within ≤5%, ≤10%, ≤15%, ≤20%, ≤25%, or ≤30%, or is the same as) the intermembrane distance spanned by the complex between a T cell receptor and its peptide-MHC antigen.

4. The immune effector cell of any one of the preceding claims, wherein the intermembrane distance spanned is about 14 nm.

5. The immune effector cell of any one of the preceding claims, wherein the height of the antigen binding domain of the CAR from the membrane of the immune effector cell is comparable with (e.g. within ≤5%, ≤10%, ≤15%, ≤20%, ≤25%, or ≤30%, or is the same as) the height of the extracellular antigen binding domain of a T cell receptor from the membrane of an immune effector cell (e.g. about 7 nm).

6. The immune effector cell of any one of the preceding claims, wherein the extracellular antigen binding domain of the CAR has a height of between about 3 nm and 7 nm from the membrane of the immune effector cell.

7. The immune effector cell of any one of the preceding claims, wherein the antigen binding domain of the CAR has a height of between about 7 nm and 47 nm from the membrane of the immune effector cell.

8. The immune effector cell of any one of the preceding claims, wherein the ligand binding domain of the accessory receptor has a height of between about 3 nm and 7 nm from the membrane of the immune effector cell.

9. The immune effector cell of any one of the preceding claims, wherein the ligand binding domain of the accessory receptor has a height of between about 7 nm and 47 nm from the membrane of the immune effector cell.

10. The method of any one of the preceding claims, wherein the height of the ligand binding domain of the accessory receptor from the membrane of the immune effector cell is about 7 nm.

11. The immune effector cell of any one of the preceding claims, wherein the accessory receptor is an endogenous accessory receptor.

12. The immune effector cell of any one of the preceding claims, wherein the CAR comprises a CD28 hinge or a CD8a hinge.

13. The immune effector cell of any one of the preceding claims, wherein the hinge of the CAR comprises or consists of a sequence that physically increases the height of the antigen binding domain from the membrane of the immune effector cell, wherein the sequence comprises a mucin-like sequence, one or more folded polypeptide domains or a fragment of the CD28 as set out in SEQ ID NO: 10 or the CD8a hinge as set out in SEQ ID NO: 43.

14. The immune effector cell of claims 1 to 12, wherein the stalk of the accessory receptor comprises a sequence that physically increases the height of the extracellular ligand binding domain from the membrane of the immune effector cell.

15. The immune effector cell of any one of claims 1 to 12, wherein the immune effector cell comprises:
(a) the accessory receptor having a stalk that comprises a sequence that physically increases the height of the extracellular ligand binding domain from the membrane of the immune effector cell; and
(b) the CAR having a hinge the comprises or consists of a sequence that physically increases the height of the antigen binding domain from the membrane of the immune effector cell, wherein the sequence comprises a mucin-like sequence, one or more folded polypeptide domains or a fragment of the CD28 as set out in SEQ ID NO: 10 or the CD8a hinge as set out in SEQ ID NO: 43.

16. The immune effector cell of any one of the preceding claims, wherein the hinge of the CAR comprises or consists of:
(a) a fragment of the mucin-like extracellular sequence of CD43 as set out in SEQ ID NO: 62, e.g. between 4 and 234 amino acids, such as between 20 and 40 amino acids (e.g. as set out in any one of SEQ ID NOs: 53 to 61);
(b) a folded polypeptide domain which is an immunoglobulin domain, such as a immunoglobulin constant domain, or a FNIII domain; and/or
(c) a fragment of the CD28 hinge as set out in SEQ ID NO: 10 or the CD8a hinge as set out in SEQ ID NO: 43, optionally wherein the fragment is as set out in any one of SEQ ID NOs: 37 to 42, 46, 63 and 64.

17. The immune effector cell of any one of claims 1 to 10 and 12 to 16, wherein the stalk of the accessory receptor comprises or consists of a fragment of a mucin-like sequence.

18. The immune effector cell of claim 17, wherein the mucin-like sequence is the mucin-like sequence of CD43 as set out in SEQ ID NO: 62, e.g. between 4 and 234 amino acids, such as set out in any one of SEQ ID NOs: 53 to 61.

19. The immune effector cell of any one of the preceding claims, wherein the accessory receptor is an adhesion receptor.

20. The immune effector cell of claim 19, wherein the adhesion receptor is CD2.

21. The immune effector cell of any one of the preceding claims, wherein:
(a) the CAR does not require endogenous TCR and/or CD3 for expression on the surface of the immune effector cell;
(b) the CAR comprises an intracellular CD32 signalling domain; and/or
(c) the CAR does not comprise an intracellular CD2 signalling domain.

22. The immune effector cell of any one of the preceding claims, wherein the CAR is a dimer, e.g. a homodimer or heterodimer.

23. The immune effector cell of any one of the preceding claims, wherein:
(a) the antigen is a peptide-MHC complex, CD19, mesothelin, BCMA, CD22, EGFR, or EGFRvIII, optionally wherein the peptide in the peptide-MHC complex is a fragment of NY-ESO 1;
(b) the ligand is CD58; and/or
(c) the antigen is different to the ligand.

24. A chimeric antigen receptor (CAR) capable of binding to an antigen on an antigen presenting cell (APC), comprising a fusion protein comprising an extracellular antigen binding domain, a transmembrane domain, and an intracellular signalling domain, wherein the extracellular antigen binding domain and the transmembrane domain are linked by a hinge, wherein the hinge comprises or consists a sequence that physically increases the height of the extracellular antigen binding domain from the membrane of the immune effector cell, wherein the sequence comprises a mucin-like sequence, one or more folded polypeptide domains or a fragment of the CD28 as set out in SEQ ID NO: 10 or the CD8a hinge as set out in SEQ ID NO: 43.

25. The CAR of claim 21, as defined in any one of claims 1 to 23.

26. An accessory receptor capable of binding to a ligand on the APC, comprising a extracellular ligand binding domain and a transmembrane domain, wherein the extracellular ligand binding domain and the transmembrane domain are linked by a stalk, wherein the stalk comprises or consists of a sequence that physically increases the height of the ligand binding domain from the membrane of the immune effector cell, optionally wherein the stalk comprises a mucin-like sequence.

27. The accessory receptor of claim 26, as defined in any one of claims 1 to 23.

28. An immune effector cell comprising or encoding the CAR and/or accessory receptor of any one of claims 24 to 27.

29. The immune effector cell of any one of 1 to 23 and 28, wherein the immune effector cell comprises a modification in its genome to reduce the expression of the endogenous accessory receptor.

30. The immune effector cell of any one of claims 1 to 23, 28 and 29, wherein the immune effector cell is a T cell, an NK cell, an NKT cell, a phagocyte or a macrophage, optionally wherein the T cell is a CD4-CD8+ T cell.

31. A method of preparing the immune effector cell, comprising introducing a nucleic acid encoding a CAR and/or an accessory receptor of any one of claims 24 to 27 into an immune effector cell.

32. A method of optimising the effector function of an immune effector cell, wherein the immune effector cell comprises:
a chimeric antigen receptor (CAR) capable of binding to an antigen on an antigen presenting cell (APC), wherein the CAR comprises a fusion protein comprising an extracellular antigen binding domain, a transmembrane domain, and an intracellular signalling domain, wherein the extracellular antigen binding domain and the transmembrane domain are linked by a hinge; and
an accessory receptor capable of binding to a ligand on the APC, wherein the accessory receptor comprises an extracellular ligand binding domain and a transmembrane domain, wherein the extracellular ligand binding domain and the transmembrane domain are linked by a stalk;
wherein the method comprises modifying the height of the extracellular antigen binding domain of the CAR and/or the height of the extracellular ligand binding domain of the accessory receptor from the membrane of the immune effector cell to optimise the effector function of the immune effector cell upon contact with the APC.

33. A method for identifying an improved immune effector cell, wherein the immune effector cell comprises:
a chimeric antigen receptor (CAR) capable of binding to an antigen on an antigen presenting cell (APC), wherein the CAR comprises a fusion protein comprising an extracellular antigen binding domain, a transmembrane domain, and an intracellular signalling domain, wherein the extracellular antigen binding domain and the transmembrane domain are linked by a hinge; and
an accessory receptor capable of binding to a ligand on the APC, wherein the accessory receptor comprises an extracellular ligand binding domain and a transmembrane domain, wherein the extracellular ligand binding domain and the transmembrane domain are linked by a stalk;
wherein the method comprises:
(a) modifying the height of the extracellular antigen binding domain of the CAR and/or the height of the extracellular ligand binding domain of the accessory receptor from the membrane of the immune effector cell; and
(b) determining whether an immune effector cell expressing a modified CAR and/or a modified accessory receptor has improved effector function when compared to an immune effector cell expressing an unmodified CAR and/or unmodified accessory receptor.

34. The method of claim 32 or 33, comprising modifying the height of the extracellular antigen binding domain of the CAR such that the intermembrane distance spanned by the CAR-antigen complex is comparable with the intermembrane distance spanned by the accessory receptor-ligand complex.

35. The method of claim 32 or 33, comprising modifying the height of the extracellular ligand binding domain of the accessory receptor such that the intermembrane distance spanned by the CAR-antigen complex is comparable with the intermembrane distance spanned by the accessory receptor-ligand complex.

36. The method of claim 32 or 33, comprising modifying the height of the extracellular antigen binding domain of the CAR and the height of the extracellular ligand binding domain of the accessory receptor such that the intermembrane distance spanned by the CAR-antigen complex is comparable with the intermembrane distance spanned by the accessory receptor-ligand complex.

37. The method of claim 36, wherein:
(a) the intermembrane distance spanned by the CAR-antigen complex is within ≤5%, ≤10%, ≤15%, ≤20%, ≤25%, or ≤30%, or is the same as, the intermembrane distance spanned by the accessory receptor-ligand complex; and/or
(b) the intermembrane distance spanned by the CAR-antigen complex is comparable with (e.g. within ≤5%, ≤10%, ≤15%, ≤20%, ≤25%, or ≤30%, or is the same as) the intermembrane distance spanned by the complex between a T cell receptor and its corresponding antigen.

38. The method of any one of claims 32 to 37, comprising:
(a) reducing the height of the extracellular antigen binding domain of the CAR;
(b) increasing the height of the extracellular antigen binding domain of the CAR;
(c) reducing the height of the extracellular ligand binding domain of the accessory receptor;
(d) increasing the height of the extracellular ligand binding domain of the accessory receptor;
(e) reducing the height of the extracellular antigen binding domain of the CAR and increasing the height of the extracellular ligand binding domain of the accessory receptor; or
(f) increasing the height of the extracellular antigen binding domain of the CAR and reducing the height of the extracellular ligand binding domain of the accessory receptor.

39. The method of any one of claims 32 to 38, comprising introducing into the hinge of the CAR and/or the stalk of the accessory receptor, or replacing the hinge of the CAR and/or the stalk of the accessory receptor with, a sequence that physically increases the height of the ligand binding domain from the membrane of the immune effector cell.

40. The method of claim 39, wherein the sequence introduced into the hinge in the CAR comprises or consists of:
(a) a fragment of the mucin-like extracellular sequence of CD43 as set out in SEQ ID NO: 62, e.g. between 4 and 234 amino acids, such as between 20 and 40 amino acids (e.g. as set out in any one of SEQ ID NOs: 53 to 61);
(b) a folded polypeptide domain which is an immunoglobulin domain, such as an immunoglobulin constant domain, or a FNIII domain; and/or
(c) a fragment of the CD28 hinge as set out in SEQ ID NO: 10 or the CD8a hinge as set out in SEQ ID NO: 43, optionally wherein the fragment is as set out in any one of SEQ ID NOs: 37 to 42, 46, 63 and 64.

41. The method of claim 39, wherein the sequence introduced into the stalk of the accessory receptor comprises or consists of a fragment of a mucin-like sequence, or a fragment or a derivative thereof, optionally wherein the mucin-like sequence is the mucin-like sequence of CD43 as set out in SEQ ID NO: 62, and optionally wherein the fragment is of between 4 and 234 amino acid residues in length, such as any one of SEQ ID NOs: 53 to 61.

42. The method of any one of claims 32 to 41, wherein the CAR, the accessory receptor and/or the immune effector cell is as defined by any one of claims 1 to 30.

43. A method for identifying an improved immune effector cell, comprising determining whether the immune effector cell of any one of claims 1 to 23 and 28 to 30 has improved effector function when compared to an immune effector cell expressing an unmodified CAR and/or unmodified accessory receptor.

44. The method of any one of claims 32 to 43, wherein the effector function is cell killing.

45. An immune effector cell obtained or obtainable by the method of any one of claims 32 to 44.

46. A method, such as an ex vivo method, of preparing a population of immune effector cells for adoptive cell therapy, the method comprises culturing the immune effector cell of any one of claims 1 to 23, 28 to 30 and 45 to produce a population of immune effector cells.

47. A population of immune effector cells produced by the method of claim 46.

48. A method for treating cancer in a subject, comprising administering an effective amount of the immune effector cell of any one of claims 1 to 23, 28 to 30 and 45, or the population of immune effector cells of claim 47, to the subject.

49. The method of claim 48, wherein the cancer is haematological malignancy or B cell cancer.

50. The immune effector cell of any one of claims 1 to 23, 28 to 30 and 45, or the population of immune effector cells of claim 47, for use in a method of treating cancer in a subject, optionally where the cancer is haematological malignancy or B cell cancer.

51. Use of the immune effector cell of any one of claims 1 to 23, 28 to 30 and 45, or the population of immune effector cells of claim 47, in the manufacture of a medicament for the treatment of cancer, optionally where the cancer is haematological malignancy or B cell cancer.

52. Use of immune effector cell of any one of claims 1 to 23, 28 to 30 and 45, or the population of immune effector cells of claim 47, to treat cancer, optionally where the cancer is haematological malignancy or B cell cancer.

53. Use of the immune effector cell of any one of claims 1 to 23, 28 to 30 and 45, or the population of immune effector cells of claim 47, for adoptive cell therapy.


SEQUENCE LISTING (66 sequences)

SEQ ID NO: 1 - protein, 133 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHP

SEQ ID NO: 2 - protein, 151 aa, synthetic construct
YIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSRAKRSGSG

SEQ ID NO: 3 - protein, 19 aa, synthetic construct
ATNFSLLKQAGDVEENPGP

SEQ ID NO: 4 - protein, 133 aa, synthetic construct
RMSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVL

SEQ ID NO: 5 - protein, 179 aa, synthetic construct
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 6 - protein, 21 aa, synthetic construct
MDFQVQIFSFLLISASVIMSR

SEQ ID NO: 7 - protein, 121 aa, synthetic construct
MAEVQLLESGGGLVQPGGSLRLSCAASGFTFSTYQMSWVRQAPGKGLEWVSGIVSSGGSTAYADSVKGRFTISRDNSKNTLYLQMNSLRAEDTAVYYCAGELLPYYGMDVWGQGTTVTVSS

SEQ ID NO: 8 - protein, 17 aa, synthetic construct
AKTTPKLEEGEFSEARV

SEQ ID NO: 9 - protein, 120 aa, synthetic construct
QSELTQPRSVSGSPGQSVTISCTGTERDVGGYNYVSWYQQHPGKAPKLIIHNVIERSSGVPDRFSGSKSGNTASLTISGLQAEDEADYYCWSFAGGYYVFGTGTDVTVLGQPKANPTVDP

SEQ ID NO: 10 - protein, 40 aa, synthetic construct
KIEVMYPPPYLDNEKSNGTIIHVKGKHLCPSPLFPGPSKP

SEQ ID NO: 11 - protein, 27 aa, synthetic construct
FWVLVVVGGVLACYSLLVTVAFIIFWV

SEQ ID NO: 12 - protein, 41 aa, synthetic construct
RSKRSRLLHSDYMNMTPRRPGPTRKHYQPYAPPRDFAAYRS

SEQ ID NO: 13 - protein, 113 aa, synthetic construct
LRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 22 aa, synthetic construct
MLLLVTSLLLCELPHPAFLLIP

SEQ ID NO: 15 - protein, 266 aa, synthetic construct
MAEVQLLESGGGLVQPGGSLRLSCAASGFTFSTYQMSWVRQAPGKGLEWVSGIVSSGGSTAYADSVKGRFTISRDNSKNTLYLQMNSLRAEDTAVYYCAGELLPYYGMDVWGQGTTVTVSSAKTTPKLEEGEFSEARVQSELTQPRSVSGSPGQSVTISCTGTERDVGGYNYVSWYQQHPGKAPKLIIHNVIERSSGVPDRFSGSKSGNTASLTISGLQAEDEADYYCWSFAGGYYVFGTGTDVTVLGQPKANPTVDPDYKDDDDK

SEQ ID NO: 16 - protein, 15 aa, synthetic construct
GGGGSGGGGSGGGGS

SEQ ID NO: 17 - protein, 185 aa, synthetic construct
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 18 - protein, 141 aa, synthetic construct
IQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSSTRGSG

SEQ ID NO: 19 - protein, 25 aa, synthetic construct
PGTMLLLVTSLLLCELPHPAFLLIP

SEQ ID NO: 20 - protein, 172 aa, synthetic construct
DLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKRKNS

SEQ ID NO: 21 - protein, 103 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 22 - protein, 5 aa, synthetic construct
GGGGS

SEQ ID NO: 23 - skipped

SEQ ID NO: 24 - protein, 106 aa, synthetic construct
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 25 - skipped

SEQ ID NO: 26 - protein, 351 aa, synthetic construct
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 27 - protein, 369 aa, synthetic construct
MSFPCKFVASFLLIFNVSSKGAVSYPYDVPDYAKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDGGGGSENSRIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 28 - protein, 373 aa, synthetic construct
MSFPCKFVASFLLIFNVSSKGAVSYPYDVPDYAKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDGGGGSRNPDENSRIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 29 - protein, 385 aa, synthetic construct
MSFPCKFVASFLLIFNVSSKGAVSYPYDVPDYAKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDGGGGSPTTSTNASTVPFRNPDENSRIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 30 - protein, 405 aa, synthetic construct
MSFPCKFVASFLLIFNVSSKGAVSYPYDVPDYAKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDGGGGSPSSGASGPQVSSVKLSTMMSPTTSTNASTVPFRNPDENSRIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 31 - protein, 415 aa, synthetic construct
MSFPCKFVASFLLIFNVSSKGAVSYPYDVPDYAKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDGGGGSPVTMTTGSLEPSSGASGPQVSSVKLSTMMSPTTSTNASTVPFRNPDENSRIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 32 - protein, 425 aa, synthetic construct
MSFPCKFVASFLLIFNVSSKGAVSYPYDVPDYAKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDGGGGSLETSTGTTGPPVTMTTGSLEPSSGASGPQVSSVKLSTMMSPTTSTNASTVPFRNPDENSRIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 33 - protein, 445 aa, synthetic construct
MSFPCKFVASFLLIFNVSSKGAVSYPYDVPDYAKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDGGGGSVSLETSKGTSGPPVTMATDSLETSTGTTGPPVTMTTGSLEPSSGASGPQVSSVKLSTMMSPTTSTNASTVPFRNPDENSRIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 34 - protein, 485 aa, synthetic construct
MSFPCKFVASFLLIFNVSSKGAVSYPYDVPDYAKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDGGGGSTITTNSPETSSRTSGAPVTTAASSLETSRGTSGPPLTMATVSLETSKGTSGPPVTMATDSLETSTGTTGPPVTMTTGSLEPSSGASGPQVSSVKLSTMMSPTTSTNASTVPFRNPDENSRIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 35 - protein, 525 aa, synthetic construct
MSFPCKFVASFLLIFNVSSKGAVSYPYDVPDYAKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDGGGGSTYQEVSIKMSSVPQETPHATSHPAVPITANSLGSHTVTGGTITTNSPETSSRTSGAPVTTAASSLETSRGTSGPPLTMATVSLETSKGTSGPPVTMATDSLETSTGTTGPPVTMTTGSLEPSSGASGPQVSSVKLSTMMSPTTSTNASTVPFRNPDENSRIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 36 - protein, 599 aa, synthetic construct
MSFPCKFVASFLLIFNVSSKGAVSYPYDVPDYAKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDGGGGSSTTAVQTPTSGEPLVSTSEPLSSKMYTTSITSDPKADSTGDQTSALPPSTSINEGSPLWTSIGASTGSPLPEPTTYQEVSIKMSSVPQETPHATSHPAVPITANSLGSHTVTGGTITTNSPETSSRTSGAPVTTAASSLETSRGTSGPPLTMATVSLETSKGTSGPPVTMATDSLETSTGTTGPPVTMTTGSLEPSSGASGPQVSSVKLSTMMSPTTSTNASTVPFRNPDENSRIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 37 - protein, 16 aa, synthetic construct
GKHLCPSPLFPGPSKP

SEQ ID NO: 38 - protein, 20 aa, synthetic construct
ENSRGKHLCPSPLFPGPSKP

SEQ ID NO: 39 - protein, 24 aa, synthetic construct
RNPDENSRGKHLCPSPLFPGPSKP

SEQ ID NO: 40 - protein, 28 aa, synthetic construct
TVPFRNPDENSRGKHLCPSPLFPGPSKP

SEQ ID NO: 41 - protein, 11 aa, synthetic construct
GKHLCPSPLFP

SEQ ID NO: 42 - protein, 6 aa, synthetic construct
GKHLCP

SEQ ID NO: 43 - protein, 45 aa, synthetic construct
TTTPAPRPPTPAPTIASQPLSLRPEACRPAAGGAVHTRGLDFACD

SEQ ID NO: 44 - protein, 24 aa, synthetic construct
IYIWAPLAGTCGVLLLSLVITLYC

SEQ ID NO: 45 - skipped

SEQ ID NO: 46 - protein, 23 aa, synthetic construct
RPEACRPAAGGAVHTRGLDFACD

SEQ ID NO: 47 - protein, 24 aa, synthetic construct
MSFPCKFVASFLLIFNVSSKGAVS

SEQ ID NO: 48 - protein, 9 aa, synthetic construct
YPYDVPDYA

SEQ ID NO: 49 - protein, 185 aa, synthetic construct
KEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLD

SEQ ID NO: 50 - protein, 5 aa, synthetic construct
GGGGS

SEQ ID NO: 51 - protein, 26 aa, synthetic construct
IYLIIGICGGGSLLMVFVALLVFYIT

SEQ ID NO: 52 - protein, 116 aa, synthetic construct
KRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 53 - protein, 4 aa, synthetic construct
ENSR

SEQ ID NO: 54 - protein, 8 aa, synthetic construct
RNPDENSR

SEQ ID NO: 55 - protein, 20 aa, synthetic construct
PTTSTNASTVPFRNPDENSR

SEQ ID NO: 56 - protein, 40 aa, synthetic construct
PSSGASGPQVSSVKLSTMMSPTTSTNASTVPFRNPDENSR

SEQ ID NO: 57 - protein, 50 aa, synthetic construct
PVTMTTGSLEPSSGASGPQVSSVKLSTMMSPTTSTNASTVPFRNPDENSR

SEQ ID NO: 58 - protein, 60 aa, synthetic construct
LETSTGTTGPPVTMTTGSLEPSSGASGPQVSSVKLSTMMSPTTSTNASTVPFRNPDENSR

SEQ ID NO: 59 - protein, 80 aa, synthetic construct
VSLETSKGTSGPPVTMATDSLETSTGTTGPPVTMTTGSLEPSSGASGPQVSSVKLSTMMSPTTSTNASTVPFRNPDENSR

SEQ ID NO: 60 - protein, 120 aa, synthetic construct
TITTNSPETSSRTSGAPVTTAASSLETSRGTSGPPLTMATVSLETSKGTSGPPVTMATDSLETSTGTTGPPVTMTTGSLEPSSGASGPQVSSVKLSTMMSPTTSTNASTVPFRNPDENSR

SEQ ID NO: 61 - protein, 160 aa, synthetic construct
TYQEVSIKMSSVPQETPHATSHPAVPITANSLGSHTVTGGTITTNSPETSSRTSGAPVTTAASSLETSRGTSGPPLTMATVSLETSKGTSGPPVTMATDSLETSTGTTGPPVTMTTGSLEPSSGASGPQVSSVKLSTMMSPTTSTNASTVPFRNPDENSR

SEQ ID NO: 62 - protein, 234 aa, synthetic construct
STTAVQTPTSGEPLVSTSEPLSSKMYTTSITSDPKADSTGDQTSALPPSTSINEGSPLWTSIGASTGSPLPEPTTYQEVSIKMSSVPQETPHATSHPAVPITANSLGSHTVTGGTITTNSPETSSRTSGAPVTTAASSLETSRGTSGPPLTMATVSLETSKGTSGPPVTMATDSLETSTGTTGPPVTMTTGSLEPSSGASGPQVSSVKLSTMMSPTTSTNASTVPFRNPDENSR

SEQ ID NO: 63 - protein, 11 aa, synthetic construct
VHTRGLDFACD

SEQ ID NO: 64 - protein, 6 aa, synthetic construct
LDFACD

SEQ ID NO: 65 - protein, 22 aa, synthetic construct
MLLLVTSLLLCELPHPAFLLIP

SEQ ID NO: 66 - protein, 9 aa, synthetic construct
SLLMWITQV